(19) United States
(12) Patent Application Publication
(10) Pub. No.: US 2026/0184650 A1
(43) Pub. Date: Jul. 2, 2026

(54) METHOD FOR CREATING A BENEFICIAL BIOFILM ON A HYDROPHILIC SUBSTRATE FOR USE AS A SOIL CONDITIONER

(71) Applicant: U.S. Biofilm L.L.C., Nashville, TN (US)

(72) Inventor: Johnny William Cude, Nashville, TN (US)

(73) Assignee: U.S. Biofilm L.L.C., Nashville, TN (US)

(21) Appl. No.: 19/545,867

(22) Filed: Feb. 20, 2026

Related U.S. Application Data
(63) Continuation of application No. PCT/US24/43264, filed on Aug. 21, 2024.
(60) Provisional application No. 63/520,895, filed on Aug. 21, 2023.
(60) Provisional application No. 63/659,788, filed on Jun. 13, 2024.

(51) Int. Cl. C05F 11/08; A01N 63/22; A01P 7/04; A01P 17/00; C05F 17/10; C05F 17/20

(52) U.S. Cl. CPC C05F 11/08; A01N 63/22; A01P 7/04; A01P 17/00; C05F 17/10; C05F 17/20

(57) ABSTRACT

The present disclosure allows for an anaerobic biofilm also known as a beneficial biofilm, to be grown on a hydrophilic substrate surface and contained within the varied surface and surface-accessible interior of the substrate. This allows for the anaerobic biofilm to remain intact and functional while being exposed to an aerobic environment. The present disclosure further allows for the constituent microbes to interact with an oxygen-rich surrounding without perishing. The present disclosure allows anaerobic biofilm constituent microbes to exist side-by-side with aerobic biofilm constituent microbes and employ various mechanisms to starve or eliminate unhealthy biofilms and sessile and free-floating unwanted microbes, and/or deliver substances to soil to improve soil health or to the body to assist in repair of injuries such as microbial infections, lesions, tumors, cuts, abrasions, burns, and nerve damage.

This application is a continuation of International Patent Application designating the U.S. serial no. PCT/US 24/43264, filed on Aug. 21, 2024, which claims the benefit of U.S. provisional patent application Ser. No. 63/659,788, filed on Jun. 13, 2024, and the benefit of U.S. provisional patent application Ser. No. 63/520,895, filed on Aug. 21, 2023, each of which is incorporated herein by reference in its entirety.

### BACKGROUND

Fusarium is an asexual fungal species that includes soil, human, and animal pathogens which often present in a biofilm matrix which is aerobic. Conventional biofilms form from microbes in a mutually beneficial syntrophic consortium. Biofilms are highly organized biological systems with one or more microorganism species organized into a coordinated functional community. Various microbes may secrete extracellular polymeric substances (EPSs) made of polysaccharides, proteins, lipids, and deoxyribonucleic acid (DNA). Within these secretions, microbes may form a living environment different from that created absent the biofilm. Resident microbes may then release secretions and biofilm-adapted progeny to the surrounding environment.

A biofilm may begin forming when a free-living bacterium attaches to a surface, such as a stent, a tooth, the intestinal tract, sewer pipes, plastic debris in the ocean, etc. A microbial cell recognizing a suitable attachment site on a surface, a quorum of likely syntrophic companions, nutritional cues such as abundant foodstuffs, etc., may change its phenotype behavior and may begin secreting various components of a biofilm. The microbe may then inhabit the biofilm and reproduce offspring exhibiting the changed phenotype behavior rather than acting as free-living microbes.

Subpopulations of cells within the biofilm may differentiate to perform various activities of the community including motility, matrix production, and sporulation. This supports the overall success of the biofilm and its ability to become further established in the host environment. Biofilm constituents may share nutrients and may be sheltered from harmful factors in the environment, such as desiccation, antibiotics, chemical treatment, environmental stresses, or a host's immune response. Biofilms may thus be more medically or environmentally harmful than their constituent microbes.

In medicine, heart stents made of smooth wire may harbor biofilms that grow much larger in size than their actual microbial constituents, clogging the stent, endangering the patient, and costing time and money to repair. Dental plaque biofilm contributes to oral and cardiac-related diseases. Biofilms containing Fusarium oxysporum, which sequesters potassium and is linked to diseases in plants and soil as well as humans, are the leading cause of medical device rejection. The dual-species biofilm that F. oxysporum forms with the yeast Candida albicans may cause infections with a 40% mortality rate. Chronic Candida infections may form aggressive biofilms when excess sugar consumption or antibiotics unbalance the body's healthy microbial communities, causing a phenomenon known as “Candida virulence.” Some species of Candida form biofilms with F. oxysporum that cause chronic biofilm-related infections that account for 80% of all cancer-related deaths. This biofilm as well as other biofilms known to cause disease are aerobic.

In nature, many soil diseases that endanger plant and animal life are caused by biofilms similar to those found in the human gut. Biofilms in the ocean containing toxic algae cause regions where seafood species caught in the area contain microbes toxic to humans. Clumps of discarded plastic have formed in the ocean due to a newly acquired ability of sargassum algae to rapidly form adhesive biofilms on smooth plastic surfaces. An increase in global temperatures may drive further detrimental changes in microbial relations in a host of environments such as the ocean, land, freshwater, air, and within and upon the bodies of all life on the planet. Biofilms which cause disease in the ocean as well as in humans are aerobic.

Probiotics provide known methods for restoring healthy gut microbiota and treating harmful biofilm infestations in soil. Conventional probiotics are used to treat unwanted aerobes in the soil, gut, bloodstream, colon, water, sewage, and industrial settings. However, they currently offer limited results since they lack a biofilm encasement. Encapsulating some beneficial microbes in biochar is known in the art with limited results. Biochar is used in agricultural settings to harbor microbes that sequester important minerals such as phosphorous. However, biochar, a naturally-produced charcoal, contains a spectrum of densities of material, as well as residues like hydrocarbons, and is known to host unwanted microbes as well. Hydrocarbon residues in biochar repel water, may cause desirable probiotic species to perish, may be toxic to soil, and, if untreated, may produce food unsafe for human consumption. Conventional methods for removing impurities in biochar exist in the art, but the processes needed for these treatments, such as removing chemical residues and gases, as well as cleaning out and widening biochar pores, may be prohibitive in cost and complexity.

A limited number of substrates for growing biofilm have been studied, such as pebbles, wood, polyvinyl chloride (PVC) and other plastics, glass, steel plumbing fixtures, and smooth metal surfaces. These substrates are generally hydrophilic. Non-smooth surfaces, such as those found in diatomaceous earth, however, resist oceanic biofilm formation. Due to their irregular surfaces, many minerals mined for industrial use, as well as volcanic dust, are found in nature to contain desirable microbes upon their varied surfaces.

Bacillus subtilis is known in the art to form an anaerobic biofilm system similar in production and action to the aerobic biofilms reported above, such as exopolysaccharide production, matrix creation, communal-driven stress resistance, and the like. However, B. subtilis may transition from a motile planktonic cell state to a sessile biofilm state to form a biofilm that is not a hydrogel, such as those reported above, but is instead an elastic sheath containing proteins and exopolysaccharides. There is, therefore, a need for the development of helpful biofilms formed within the protein matrix supplied B. subtilis.

### BRIEF SUMMARY

The present disclosure includes a method of preparing a biofilm. The method includes treating a bare hydrophilic substrate with a saline solution, adding the treated substrate to a nutrient solution, adding microbes to the nutrient-substrate mixture, incubating the microbial mixture, resulting in an inoculated substrate with a beneficial biofilm, and harvesting inoculated substrate with the beneficial biofilm.

The present disclosure further includes a method for treating a target surface with a biofilm by preparing the biofilm as described above and applying the biofilm to the target surface by at least one of spraying, dropping, scattering, and dusting the target surface.

Finally, the present disclosure includes the biofilm prepared and used as described above.

### BRIEF DESCRIPTION OF THE DRAWINGS

To easily identify the discussion of any particular element or act, the most significant digit or digits in a reference number refer to the figure number in which that element is first introduced.

FIG. 1 illustrates an activated charcoal particle 100 in accordance with one embodiment.

FIG. 2 illustrates a mineral particle hydrophilic substrate 200 in accordance with one embodiment.

FIG. 3 illustrates diatomaceous earth particles 300 in accordance with one embodiment.

FIG. 4 illustrates a beneficial biofilm 400 in accordance with one embodiment.

FIG. 5 illustrates a process 500 in accordance with one embodiment.

FIG. 6 illustrates a process 600 in accordance with one embodiment.

FIG. 7 illustrates a process 700 in accordance with one embodiment.

FIG. 8 illustrates a process 800 in accordance with one embodiment.

FIG. 9 illustrates a process 900 for treating a target surface with a biofilm in accordance with one embodiment.

### DETAILED DESCRIPTION

The biofilm system of the present disclosure may utilize probiotic microbes (microbes which are known to be of benefit to life) contained within a semi-closed localized environment such as a non-smooth hydrophilic substrate. The disclosed biofilm is anaerobic. When placed within an aerobic environment, it may compete with and destroy aerobic biofilms which cause disease. The essential substrate(s) which may be employed to create the anaerobic biofilm have hydrophilic surfaces. During creation of the anaerobic biofilm matrix, the surface is coated with a durable protein matrix supplied by Bacillus subtilis.This protein matrix forms an envelope for containing all the microbial constituents.

The present disclosure allows for an anaerobic biofilm also known as a beneficial biofilm, to be grown on a hydrophilic substrate surface and contained within the varied surface and interior of the substrate. This allows for the anaerobic biofilm to remain intact and functional while being exposed to an aerobic environment. The present disclosure further allows for the constituent microbes to interact with an oxygen-rich surrounding without perishing. The present disclosure allows anaerobic biofilm constituent microbes to exist side-by-side with aerobic biofilm constituent microbes and employ various mechanisms to starve or eliminate the unhealthy aerobic biofilm.

Anaerobic biofilms differ from aerobic biofilms in that they occur in anaerobic environments and are made of constituents that prefer a carbon dioxide-rich environment rather than an oxygen-rich environment. Further, anaerobic biofilms differ in the surface upon which they attach. Anaerobic biofilms prefer porous hydrophilic surfaces, whereas aerobic biofilms prefer hydrophobic surfaces which may be smooth.

Preparatory techniques described herein may influence the constituent microbes of the present disclosure to prefer a hydrophilic surface environment and to employ methods for survival and for eliminating aerobic biofilm constituents that probiotic microbes not born in a biofilm environment may not employ.

The beneficial biofilm processes described in the present disclosure may be made by incubating an anaerobic biofilm upon a mixture of different hydrophilic substrates. By extension, the result of the component materials may then be transferred to a recipient or to a sympathetic environment whereupon the present disclosure is made manifest.

The varied surface of the substrate material may have a topography that has cavernous spaces, pits, valleys, vacuoles, folds, recesses, indentations, holes, and/or semi-enclosures contained within that are accessible from the surface. The biofilm coating the substrate is able to occupy the varied surface terrain and semi-enclosures within the substrate. As a result, the substrate and protein coating of the biofilm comprise the outer covering of an anaerobic environment, allowing for the anaerobic syntrophic microbial community to live within.

On the substrate exterior, the varied terrain along with a coating of BsIA bacterial hydrophobon protein creates an anaerobic environment on the surface allowing for safe attachment and habitation by the constituent microbes of the present disclosure which include anaerobic microbes as well as facultative microbes and obligate aerobes all living within the enclosure. BsIA protein, produced by B. subtilis, covers the enclosure. The resulting portable substrate system may be deposited in or upon an environment that is aerobic while maintaining the constituent microbial community's localized anaerobic environment, thereby protecting and partitioning constituents of the present disclosure from the otherwise aerobic harmful environment.

One manner of making the beneficial anaerobic biofilm may be to simply combine all of the microbes in a single inoculant. Hydrophilic substrate material such as charcoal, diatomaceous earth, etc., may be added. After a period of incubation, the beneficial biofilm of the present disclosure may occur upon the surface of the substrate and within interior spaces of substrate particles that are accessible to the surface. The beneficial biofilm may also occupy a portion of the remaining liquid. The beneficial biofilm may be transferred as a liquid, as a slurry containing both liquid and substrate, as a filtrate, as a powder, as a pill, as a pellet, or through other methods known in the art.

Another manner of manufacture involves creating at least two or more inoculants, one containing anaerobic and facultative microbes and one containing obligate aerobic species with a companion species. Each incubation inoculate may be placed in a separate bioreactor with a hydrophilic substrate. After a period of incubation, the remaining materials may be held apart and combined upon a recipient or sympathetic environment at a later date. After a period of incubation on the recipient, the present disclosure is made manifest.

Another manner of creating the present disclosure involves creating multiple inoculants made of various combinations of the constituent microbes, containing any combination of one or more of the anaerobic, facultative, or obligate aerobic species. Each may be placed in a bioreactor with a hydrophilic substrate. The substrate may differ in any of the incubation systems presently described. After a period of incubation, the material may be harvested from individual bioreactors. The remaining materials may be held apart and combined upon a recipient or sympathetic environment at a later date. After a period of incubation on the recipient or sympathetic environment, the present disclosure may be made manifest.

A further method of creation of the present disclosure involves adding other species types to the constituent microbes to form the inoculant in order to expose the constituent microbes to foreign microbes which may or may not be hostile and thereby may enhance the biofilm system of the present disclosure.

The biofilm inoculant(s) is/are a mixture of probiotic microbes that are generally regarded as safe for human consumption and are thought to restore a healthy gut biome, encourage healthy plant growth, and contain anaerobic, facultative and obligate aerobic species. Constituent inoculants may contain any number of the groups of microbes named herein. In one embodiment, constituents may include Lactobacillus acidophilus (from yogurt), Lactobacillus plantarum (from sauerkraut and kimchee), Bacillus subtilis (from kimchee), Rhodopseudomonas palustris (purple non-sulfur bacteria), and actinomycetes (from edible probiotics such as EM-1 from Tera Ganix, Corp. The components may be grown in nutrient solution(s) forming an inoculant that may be combined with a hydrophilic substrate. The hydrophilic substrate may have a non-smooth, variable surface having one or more of the following features: cavernous spaces, pits, valleys, vacuoles, folds, indentures, holes, and/or enclosures accessible from the surface. This process is called biofilm incubation.

After a period of incubation, the result is a slurry containing substrate material inoculated within and upon its topography with an anaerobic biofilm in combination with a resulting mature inoculant liquid containing the anaerobic biofilm in liquid form.

The beneficial biofilm system of the present disclosure has applications in agriculture, industry, aquaculture, coral reef restoration, food, and medicine.

When the present disclosure is placed upon a recipient environment that is aerobic, constituent microbes of the present disclosure employ a variety of methods for successfully encountering and influencing the aerobic surroundings. Strategies for inhabiting the aerobic environment may include creating progeny which upon release from the biofilm enclosure quickly perform autolysis, thereby depositing their contents on the recipient habitat. This method may be employed to feed other microbes simultaneously being released from the anaerobic enclosure of the present disclosure. Microbes of the present disclosure may release progeny onto a recipient environment that undergo the act of autolysis, thereby depositing secretions on the new territory in order to deny or limit the encroachment of foreign microbes stationed in the new habitat.

The present disclosure comprises microbes in an anaerobic environment that rely on the substrate and biofilm system of the present disclosure in order to live in an aerobic surrounding. Facultative microbes as well as obligate aerobes of the present disclosure are equipped with the ability to live in either aerobic or anaerobic environments under certain conditions and may undergo changes in gene expression and successfully inhabit an aerobic environment as a free-living microbe.

The present disclosure contains some microbial species which are known to be facultative or obligate aerobes, are oxygen-tolerant, and when placed in a recipient environment that is aerobic, may receive and transmit signals to produce progeny better equipped to tolerate the recipient environment.

Microbes of the present disclosure which are oxygen-tolerant microbes may respond to a recipient environment containing oxygen and may signal some communal constituents within the biofilm to deploy strategies to reduce or eliminate oxygen exposure to some or all microbial constituents. Oxygen-tolerant constituents may produce protective secretions within and around the exterior openings or within and around regions of the biofilm where anaerobes are housed to seal the localized regions as a way to reduce oxygen penetration into the biofilm so as to protect the anaerobic cohorts.

Oxygen-tolerant species of the present disclosure may resort to producing progeny that is not released from the present disclosure but remains intact within the confines of the biofilm of the present disclosure serving to encase resident anaerobes and further, may retract or deploy from the biofilm during periods of reduced oxygen penetration, and may be dissolved by means of enzymatic secretions and other methods known by a person skilled in the art. This strategy of producing progeny to protect biofilm residents is herein termed facultative end-capping.

This strategy of oxygen-tolerant species of the present disclosure to engage in oxygen-shielding by means of capping off an environment is especially useful in clay-rich topsoil, where dry periods may cause increases in surface air penetration due to cracks in the soil, whereas wetter periods may serve to reduce penetration by surface air. This cycle may occur daily during dry periods when morning dew is heavy or between periods of rain or watering.

The present disclosure may be deposited upon an area that is anaerobic. This may result in a deployment of the progeny of the present disclosure onto the agreeable environment where the constituent microbes establish similar anaerobic biofilm systems to their parents by methods described herein. This process may be established in the case of yard waste or landscape waste where the material is treated with the present disclosure and a tarp is placed over the material to establish a recipient environment that is anaerobic. The same may be employed by someone desiring to decompose fallen trees, food waste, manures, byproducts of the slaughter industry, and other waste products having a composition capable of providing the foodstuff of the present disclosure and held in an enclosure that eliminates exposure to air like tarps or mulching, and under concrete, or asphalt, where it may prevent sinkhole formation.

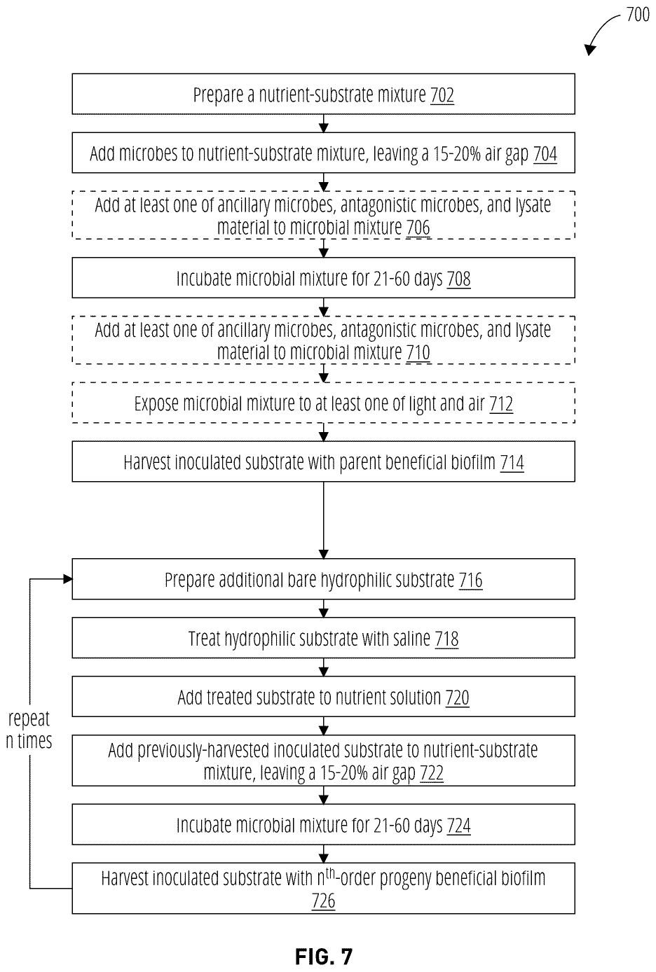

If an area is newly covered in water, such as a plant whose roots have become flooded or a plant whose roots penetrate into an area of clay-rich soil that retains water, resident microbes of the present disclosure may be able to deny or starve out formations of aerobic biofilms which are opportunistic in such settings. Additionally, a version of the biofilm system of the present disclosure, described with respect to FIG. 7 as ancillary microbes, may incorporate Lactobacillus plantarum, which may decompose lignin in the soil, provide nutrients and CO₂ to the plant, and reduce clay deposits, thus providing drainage.

A further theory regarding the present disclosure postulates that the introduction of ancillary species to the inoculant of the present disclosure, which may support the biofilm of the present disclosure, such as the addition of Lactobacillus plantarum, having been demonstrated as capable of breaking down lignin, absorbing nutrients, deny harmful aerobes and sequestering iron for constituent microbes of the present disclosure, may lead to an arrangement of the biofilm that differs from biofilms of the present disclosure that do not contain the ancillary microbe. Such phenomena may be dependent on the nutrients in the recipient environment. Lignin is particularly difficult to decompose without the use of a version of the present disclosure containing Lactobacillus plantarum and known herein as ancillary microbes. Recipient environments having localized concentrations of lignin may inspire versions of the present disclosure extremely rich in Lactobacillus plantarum and have an overall biofilm arrangement that differs from other versions of the present disclosure.

Resident microbes of the present disclosure may undergo synthesis of various compounds resulting in the releasing or off-gassing of carbon dioxide gas into the local soil, for uptake and the creation of free space in the soil, thereby increasing the soil permeability, resulting in the elimination of trapped water, and inviting roots to form, as well as creating habitat for other beneficial anaerobic microbes.

Ponds, aquariums, drip lines, and water troughs contain oxygen-rich water and often contain a slimy aerobic biofilm. The present disclosure deploys a number of strategies to eliminate the aerobic biofilm including releasing protozoa which rapidly uptake nutrients and foodstuff, thereby starving the constituents of the aerobic biofilm and eventually eliminating it.

While not being held to a theory, it's presumed that resident microbes of the present disclosure do not deploy from their biofilm encasement and may remain housed indefinitely; however, they do produce progeny that may rearrange their positions within the present disclosure over time to accommodate changes in the environment, communal demands, exposure to chemicals such as antifungals, for reasons familiar to anyone in the art or due to reasons unforeseen at the present.

In one embodiment, a hydrophilic substrate is coated with an anaerobic biofilm and is transferred to a recipient environment to cause the formation of an anaerobic biofilm for the purpose of releasing carbon dioxide.

In one embodiment, a hydrophilic substrate is coated with an anaerobic biofilm and is transferred to a recipient environment to cause the formation of an anaerobic biofilm for the purpose of denying harmful, disease-causing microbes.

### Biofilm Theory

The present disclosure is capable of producing an anaerobic biofilm in a recipient environment. The device described herein is able to deposit a biofilm comprised of a group of syntrophic microbes, known to be probiotic, found to be beneficial, are grown in a media comprising a microbial inoculant, inhabit a substrate, live within an extracellular polymeric substance (EPS) and are coated with a protective BsIA protein. The substrate material along with the protective outer biofilm coating are capable of sheltering and encasing the enclosed beneficial microbes. The constituents inhabit a mostly anaerobic environment and remain intact within the substrate and protective coating. Constituent microbes remaining intact are able to produce progeny which may produce secretions capable of modifying the enclosure and are able to exit while other constituent microbes that remain in the biofilm are able to repair the opening by secretions, progeny, or methods well known in the art.

After incubation, the resulting mature incubation liquid and the fully inoculated substrate known herein as parent biofilm may be utilized as parents for producing a second generation of biofilm inoculated substrate material. The benefit of creating a second inoculant which is supplied with microbes grown within a biofilm matrix offers a more organized system for creating a resilient anaerobic biofilm due in part to the fact that the progeny were conceived, lived in, and departed from a communal biofilm environment rather than having been the progeny of microbes of a free-living lifestyle. The resulting biofilm which occurs on a hydrophilic substrate is denoted herein as simple progeny. Using the resulting progeny on a newly inoculated substrate as the inoculating microbes for a successive generation is a means for conditioning constituent microbes to the communal state of biofilm formation and away from the lifestyle of a free-living microbe and producing progeny that seek to form a biofilm.

Without being held to a theory, it is speculated that utilizing an inoculant that contains a ready-made biofilm matrix as the source for the microbes to be incubated, known herein as simple progeny is a process that further reduces stress on the microbes and creates a second progeny more adept at forming a biofilm matrix. For example, some constituents of the present disclosure form protozoa which may be deployed from the biofilm matrix and into a recipient environment. It is herein proposed that the number of protozoa and the physical localized position of protozoa within the biofilm matrix in connection to their escape route from the biofilm enclosure may be modified with each successive generation of progeny created from a biofilm of the present disclosure, to ensure a more successful deployment onto a recipient environment. Each generation of progeny grown from a lineage of anaerobic biofilm ancestors has encoded within their gene expression the biofilm-forming phenotype and more readily scavenge for the items and perform the actions needed to form an anaerobic biofilm, such as seeking foodstuffs, quorum sensing, locating hydrophilic substrates, and explorations to find localized regions that may foster an anaerobic environment and protect other constituent microbes which may need protection, rather than behave as self-serving free-living microbes.

Without being held to a theory, it is speculated that utilizing an inoculant that contains a ready-made biofilm matrix that is directed to ample foodstuffs and hydrophilic substrate material, results in a generation familiar with biofilm formation and its mutual benefits such as reduced stress on the constituents. Such a process anticipates the rewards of quorum-sensing and biofilm formation, employs phenotype changes having undergone reduced stress on the constituent microbes as the process anticipates the need for quorum sensing, is readied for phenotype changes, and creates a next-generation progeny more adept at forming a biofilm matrix having found the path of least resistance, which includes conservation of energy which may present as a faster, more abundant productions of progeny, protection supplied by other constituents, and other benefits described herein.

It is further imagined that a progeny biofilm of the present disclosure may be better prepared for success in a recipient environment if the inoculation process includes stresses brought about by the influence of exposure to causative events such as variations in light, heat, air, and or oxygen in a biofilm, noted herein as modified progeny. In a manner similar to how computers may facilitate machine learning, biofilms may encode stresses and later inform responses to those stresses, or biofilms described herein may encode the presence of unwanted microbes or competing biofilms based on conditions applied during their formation, so that they are already prepared to perform a desired function, such as how various constituents arrange themselves, seek out foodstuffs, and seek out anaerobic niches and useful substrates.

A constituent microbe of the present disclosure is heat-loving as well as light-loving and increases in number among its cohorts when heat and or light greater than that which is normally supplied to the present disclosure occurs. Incubation periods that involve increases in heat and or light greater than normal, result in a version of the present disclosure with greater numbers of the heat-loving and light-loving species within the biofilm matrix. Incubation periods that involve increases in air and or oxygen greater than normal result in a version of the present disclosure with greater numbers of obligate aerobes and facultative species within the biofilm matrix.

By extension, a modified progeny containing an increase in the amount of a heat-loving and light-loving species of the present disclosure creates a version of the present disclosure more capable of living near the surface of a recipient environment where greater amounts of heat and or light are anticipated. Therefore, soil may be made more heat-tolerant.

An embodiment of the present disclosure describes a pellet manufactured for distributing the present disclosure in a recipient environment. The pellet may be constructed containing versions of the present disclosure designed for distribution in that recipient environment, including versions that are more adept at dealing with exposure to higher-than-normal levels of light or heat. These versions may be incorporated into the same pellet with a version of the present disclosure formerly described as ancillary microbes, which may contain lactobacillus plantarum, designed to break down lignin in the soil or clay deposits, having the resultant effect of treating multiple issues in a single pellet composition.

The inoculated and fully incubated substrate system of the present disclosure may be deployed upon a recipient environment while the constituent microbes do not deploy from the substrate but do produce progeny, lysates, and secretions that are released through openings in the protective protein coating and deposited onto the recipient environment.

The present disclosure is incubated in an anaerobic environment and is therefore labeled herein as an anaerobic biofilm. The constituent microbes are grown in a media made of nutrients, minerals, and foodstuffs comprising an anaerobic biofilm inoculant. To the inoculant is added a hydrophilic surface material that has been pre-treated with a saline solution to create a uniform hydrophilic surface. Increasing the amount of sodium chloride in the inoculant of the present disclosure may create a resultant progeny which is modified in such a way as to increase the success of the resulting biofilm in its ability to live in salt-rich environments such as estuaries, salt lakes, oceans and other environments near the ocean.

### Lab Techniques

The biofilm system may be first generated from microbes that are incubated in media that also contains a hydrophilic substrate material. Adding the hydrophilic substrate to the constituents of the inoculant summons the microbes to live in a new environment. The progeny from this step may encode info about their success in the new environment. During this initiating step, microbes go from living free of a hydrophilic surface to inhabiting a hydrophilic surface. This first-generation progeny may be used as a simple version of the present disclosure, or it may be used as the microbial source in a biofilm inoculant. The hydrophilic substrate material harvested from this parent inoculant which contains the first-generation biofilm progeny is a more ideal starting material for the disclosure.

In the present disclosure, the parent generation which is living on the hydrophilic substrate and in the mature liquid inoculant (also referred to as the supernatant) may be combined in a slurry, used as a filtered supernatant, or used as a dried filtrate material to supply the microbial constituents which are added to the inoculant mixture to initiate a new biofilm inoculation.

The process of preparing a hydrophilic surface involves selecting the hydrophilic substrate(s) from a known list of useful material, washing the substrate material with water or chemicals, or combining it with mature biofilm inoculant of the present disclosure, rinsing the substrate with water, drying it, and placing it in water and adding salt (NaCl) to create a 0.9% saline solution at a pH of 5.5. The substrate is filtered, drained, added to a newly created nutrient solution and water mixture, brought to 41° C., and stirred enough to incorporate all the ingredients. The next step involves adding the microbial media at 41° C. The microbes may be sourced from free-living microbes or any combination of free-living microbes and substrate-bound microbes produced by the present disclosure. Stirring slowly for 15 minutes is essential. Fast stirring may bruise or shear the microbes. The mixture may be stirred slowly for a few minutes each day until completion. The solution may cool to 32° C. and remain at that temperature in a hotbox for 50 days to completion. If the solution cools to 21° C., it may take 60 days to completion.

The biofilm inoculation may occur in a sealed container with an air gap of, for example, 20% of the entire volume of inoculant. The air gap supplies a portion of oxygen, carbon dioxide, and nitrogen gas, which may be essential for the growth of some of the constituents. In another embodiment, the gases needed may be supplied into a system without an air gap, with an outlet valve allowing excess gas to escape. Alternatively, an inlet valve may be affixed to the bioreactor vessel which may compensate for the lack of an air gap by allowing gas exchange. An air gap larger than 20% of the inoculant volume may also be used with the gases needed supplied through a hose running into the liquid within the bioreactor. Excess air above the surface of the liquid may be purged using an inert gas or a gas that is not reactive to the biofilm system. Gases supplied through this arrangement may contain some quantity of gases useful to the biofilm along with some quantity of non-reactive or inert gases. The proportions of such a mixture may be varied as needed to achieve the desired resulting biofilm, noting that excesses of any gas may destroy the microbes therein.

The saline solution may be made with ordinary table salt. In one embodiment, Himalayan salt or sea salt may be used, each of which contains trace metals and other minerals important for the biomanufacture of certain enzymes.

Water used may need to be free of chlorine and free of contaminants. It may contain minerals such as iron and may contain trapped gases.

### Handling Directions

Sanitation is important. For medical applications, precautions may be taken to perform the work in a vent hood to eliminate the effects of human breath and to eliminate foreign microbes from contaminating the media of the system.

A number of factors may influence the effectiveness of the media, influence the biofilm inoculation process, and cause microbial stress, such as pH, temperature, the amount of oxygen at the initiation of inoculation, dissolved CO₂, chemicals, microbial lysates, the shear rate of the stirring mechanism, the seal of the container, the concentration of microbes, the concentration of substrate material, and other factors one of ordinary skill in the art will appreciate.

The media may be composed of nutrients capable of feeding the microbes of the present disclosure, as well as any other added microbes or cells, as described herein.

### Substrates

Hydrophilic substrate material types ideal for the present disclosure fall into three categories: inert, perishable, and living surfaces. Inert materials are defined herein as those which do not degrade or may degrade very slowly when placed in soil under normal environmental conditions due to, for example, water erosion. Such materials include activated charcoal, biochar treated by methods disclosed herein, bone, charred bone, crushed shell, diatomaceous earth, perlite, particles of trace minerals, granite, quartz, gypsum, kaolinite, cassiterite, hematite, limestone, paramagnetic rock dust, natural clays, manmade clays, hydroxyapatite, nanocomposite tissue scaffolds for bone repair, porcelain, or any hydrophilic substance that is inert and does not do harm to the soil or cause contaminants to accumulate in food grown in the soil. The materials contained herein that do not degrade in soil may create inoculated substrate of the present disclosure that permanently releases progeny into a recipient environment.

Perishable substances useful as hydrophilic substrate material for the present disclosure include grain, crushed grain, bran, grain hulls, wood, nuts, nut hulls, cellulose waste, paper, tree bark, leaves, yard waste, scorched soil, and any hydrophilic material which breaks down in soil and does not do harm to the soil or cause contaminants to build up in food grown on the soil. Additional perishable surfaces that are hydrophilic and suitable for the present disclosure include surgical thread, surgical bandages, diapers, surgical gowns, bed sheets, or any hydrophilic material used in the medical industry.

Any perishable material that is used as substrate material in the present disclosure may also contribute foodstuffs to the microbes of the present disclosure and may incur some degree of decomposition during the inoculation process of the present disclosure.

Living surfaces suitable as hydrophilic substrate material for the present disclosure include hydrophilic regions of the body containing epithelia cells such as skin, both inside and outside the human body, any region along the alimentary canal such as the mouth, throat, gut, intestines, anus, or rectum, as well as hair, nails, teeth, open wounds, internal organs, bone, bone marrow, stem cells, embryos, and cartilage.

Hydrophobic materials may be treated to become hydrophilic with the addition of a surfactant. Any material may be modified physically to enhance its surface in a way that increases the hydrophilic quality of the surface thereby making it a more suitable candidate for the present disclosure. Smooth surfaces favor hydrophobicity, whereas non-smooth surfaces favor hydrophilicity. Therefore, surface modifications may be made to prevent a smooth surface from forming or to modify a smooth surface into a non-smooth surface. For example, a surface may be altered or designed to include pits, pores, valleys, indentures, and or other surface modifications described herein, thus becoming useful substrates for the present disclosure. Materials that are conventionally created having a smooth surface, but which may be modified to be non-smooth for use as substrate material for the present disclosure include, but are not limited to: smooth metal surfaces like surgical steel used in making surgical suites like scalpels, clamps, cabinets, tables, and devices such as surgical hip implants; smooth metal surfaces used in injection moldings for forming metal, composite, plastic, or glass items, including plastic items made from the plastic injection molding process, metal wire used in stents or corrective braces for the teeth, glass for catheters and tools or containers used in harvesting, handling, or storing anaerobic cells such as anaerobic microbes, anaerobic biofilms of the present disclosure, bone marrow, stem cells, and embryonic cells; smooth metal surfaces used for plowing, disking, and tilling soil; composite metal surfaces; composite non-metal surfaces; the smooth surface of plastics made in sheets or films; fiberglass structures; and building materials. Metal surfaces may be made hydrophilic and therefore useful to the creation of the present disclosure if they are etched by lasers, water, plasma, and the like, or by mechanical means such as scratching, scarring, sanding, and tumbling. Laser created substrates and chemically etched substrates may contain modifications that appear subtle and remain smooth to the touch, yet contain valleys, pores, pits, and other surface characteristics named herein that may mimic naturally-occurring hydrophilic substrates. Such surfaces may serve to both accompany the present disclosure and prevent unwanted microbes, such as the surface of limestone, which is a useful substrate for the present disclosure and has a surface known in the art to prohibit the growth of Escherichia coli(E. coli). Items such as surgical equipment, vehicles meant for space travel, clean rooms, surgical suites, and items that are meant to avoid unwanted microbial infiltration may be submitted to an inoculation process for a number of cycles in order to build a thicker beneficial biofilm layer upon the surfaces than a single cycle would deposit.

Biochar is a remnant of burning wood, is hydrophobic, and is not useful for the present disclosure unless modified. Biochar is a common material that differs from activated charcoal in that it often contains residues comprised of any combination of chemicals, biological agents or inert material. Biochar may be modified to be useful for the present disclosure by combining the biochar with inoculant material of the present disclosure which includes a nutrient solution, constituent microbes, and a hydrophilic substrate. All of the aforementioned materials, including the biochar, may be enclosed inside a bioreactor with a 20% air gap. The bioreactor may be equipped with a gas exchange valve or valves which may serve to exchange an amount of gas into and out of the system. Such gases may include air, oxygen, inert gases, carbon dioxide, other gases found in air such as neon, and any gas useful for creating the present disclosure. The biochar combined with the materials normally used to create the present disclosure, as described herein, may then be subjected to the normal conditions inside the bioreactor that occur during the inoculation processes of the present disclosure. These conditions may include mechanical stirring, heat, increased air pressure caused by the activity of the constituent microbes inside the sealed container, a period of vacuum that occurs at the end of the inoculant cycle due to activities of the constituent microbes in a sealed container, and, in some cases, added amounts of any combination of heat, light, air, oxygen, and additional nutrients. The resulting treated biochar may be modified in various ways familiar to anyone of ordinary skill in the art, such as widening of pores, deepening of pores, and removal of residues that may be chemical, biological, inert, or any combination thereof. The resulting treated biochar may be harvested and dried and may be useful for the present disclosure. The treated biochar may provide a hydrophilic surface suitable for growing the present disclosure, though it may in some cases need to undergo multiple cycles of exposure to the biofilm inoculation process before it is suitable. When certain microbes that are constituents of the biofilm disclosed herein are exposed to repeated cycles of growth on hydrophilic surfaces (pores), there is an increase in the formation of flagella (tails) as opposed to the formation of a slime. The resulting treated biochar may need a simple wash and a treatment with saline to be useful for the present disclosure. Successfully treated hydrophilic materiel may further need to be sterilized, and thus may undergo a heated wash at a temperature capable of sterilizing the resulting treated biochar, destroying the microbes contained within and upon the biochar surface, then may be harvested, dried, and treated with a 0.9% saline solution to be useful for the present disclosure.

Some materials, such as corn, other seeds, or high-density hardwoods, may have a hydrophobic surface that is easily modified and may be altered to become hydrophilic by placing it into a mixture of the inoculant solution along with the useful substrate of the present disclosure. Easily modified surfaces such as those mentioned herein may be added to an ongoing biofilm inoculation at any point in time during the inoculation process of the present disclosure and may be removed once the surface becomes hydrophilic. Such material may then be a hydrophilic substrate useful for the creation of the present disclosure.

A hydrophilic surface or a hydrophobic surface may be modified or created to be non-smooth and more suitable for the present disclosure. For example, the surface of an injection mold may be altered so that it leaves an imprint on the object created, providing an enhanced surface of valleys, pits, pores, and indentures, as well as other modifications described herein. This may cause items created from the modified injection mold to have a non-smooth surface, making them more useful as a substrate for the present disclosure. Such items may further benefit from the addition of a surfactant.

### Nutrients

Nutrient materials used to make the inoculant media (i.e., nutrient solution) used to feed the constituent microbes of the present disclosure during the inoculation process may include but are not limited to molasses, fish paste, fish emulsion, blood, bloodmeal, fermented shrimp, rice bran, wheat bran, fermented yams, sea salt, granulated kelp, vitamins, soy flour, malic acid, quinoa flour, polysorbate 80, agar, minerals, and any material useful for growing the constituent microbes of the present disclosure, as will be appreciated by one of ordinary skill in the art. Minerals include but not limited to sea salt, magnesium oxide, iron oxide, potassium oxide and phosphorous pentoxide.

### Microbes

Bacillus subtilis is a Gram-positive bacteria known to be a plant-growth regulator used in the art. B. Subtilis is known to supply enzymes for protein production useful to plants, animals and for use in biosynthesis. Varieties of B. Subtilis suitable for the present disclosure may synthesize or produce pulcherrimin, synthesize and secrete an exopolysaccharide and an amyloid fiber-forming protein TasA, and assemble surface regions with BsIA protein.

B. subtilis exists in many forms. It has undergone significant manipulation in the bioengineering sciences where it is often employed for its rich source of enzymes and useful biological agents for biosynthesis. Wild-type strains of B. subtilis may be found in fermented foods such as kimchee or natto. One of ordinary skill in the art will appreciate that B. subtilis strain QST 713 is sold as a common biofungicide and expresses all of the characteristics useful in the present disclosure. Without limits, any strain of B. subtilis which does not supply all of the characteristics mentioned herein as useful, such as a hydrophobon that forms a BsIA protein biofilm coating, pulcherrimin, EPS, or TasA, is not a useful candidate for the present disclosure, but may be used as a useful ancillary microbe as described herein.

Pulcherrimin is a useful chemical substance in the present disclosure as it limits the size and amount of biofilm formation in the present disclosure. As a result, pulcherrimin formation may be brought about by many different microbes other than B. subtilis, which may be added as an ancillary microbe by methods described herein.

Pulcherrimin formation is essential in the present disclosure as its presence limits the growth of the biofilm culture of the present disclosure. Pulcherrimin has been found to be antibacterial and it is a method for storing iron for later use by other constituent microbes of the present disclosure. When used as an additive to watering, B. subtilis varieties used in the present disclosure promote robust, healthy growth and stress resistance, enhance photosynthesis, and kill soil disease-causing vectors such as Pythium, Rhizoctonia, and Fusarium.

As a foliar, B. subtilis strains used in the present disclosure protect against Botrytis, Powdery mildew, Sclerotinia, Xanthomonas, and Erwinia. B. subtilis has a growth cycle of around 48 hours. Strains of B. subtilis useful in the present disclosure are generally regarded as safe (GRAS).

The photosynthetic bacteria used for the present disclosure may be Rhodopseudomonas palustris. Useful ancillary bacterial of this type may include members of the family Rhodospirillaceae such as R. rubrum, R. tenue, R. fulvum, R. molischianum, R. photometricum, Rhodopseudomonas gelatinosa, R. capsulata, R. viridis, R. acidophila, R. sphaeroides, R. vannielii, etc.; members of the family Chromatiaceae such as C. vinosum, C. okenii, C. warmingii, C. bunderi, C. minus, C. violascens, C. weissei, C. gracile, etc.; and Thiocystis galatinosa, T. violacea, Thiospirillum sanguineum, T. jenense, T. rosenbergii, etc.; and the like. Among others, so-called purple non-sulfur bacteria belonging to the genera Rhodospirillum and Rhodopseudomonas may be most beneficial. Moreover, two or more species or strains of these bacteria may be used to better effect than a single species or strain, because they may form a more stable mixed system with an actinomycete. Strains of photosynthetic bacteria useful in the present disclosure are generally regarded as safe. The incubation cycle of photosynthetic bacteria is dependent on the availability of organic acids and the availability of an energy source such as light, heat, or an electromagnetic field, e.g. a magnetic field. Given the rich temporal change that exists in the complex and ever-changing daily environment of the present disclosure, it is proposed that photosynthetic microorganisms of the present disclosure both adapt to and contribute to these daily dynamics through the process of temporal mutualism, and therefore biofilm systems of the present disclosure may differ depending on amount and type of nutrients, microbes, conditions, and other factors described herein. However, it is understood by one of ordinary skill in the art that the largest bloom of photosynthetic bacteria occurs after about 15 days under normal conditions, and further, may be brought about more abundantly with increased exposure to light, air, or a feeding regiment which adds or produces an increase in organic acid formation.

The present disclosure may use any member of the Actinomycetes family that is of the genera Actinomyces, is non-pathogenic, is regarded as safe, produces and secretes lactic acid, is able to tolerate the low pH (3.7) encountered during incubation, and can live within the biofilm system of the present disclosure. These may commonly be found in soil, in mineral mines, or in fully fermented compost. Other such actinomycetes that may be ancillary cohorts as described herein include, for example, members of the genera Nocardia, Thermomonospora, Micromonospora, Pseudonocardia, Chainia, Streptomyces, Actinoplanes, Streptosporangium, and Agromyces.

The microorganisms of the present disclosure are contained within the BsIA biofilm of the present disclosure and further inhabit the porous regions of the hydrophilic substrate described herein. The progeny of these microbes compete with the harmful microorganisms present in an environment such as the human gut, skin, water, and soil and utilize them as nutrient sources to eliminate the detrimental effects thereof. In this manner, they themselves propagate as useful microorganisms or facilitate the propagation of other useful microorganisms in an environment, thereby promoting a healthy environment.

Accordingly, it is to be understood that any of the actinomycete and photosynthetic bacteria used as ancillary microbes as described herein may be sourced from genera other than the above-enumerated genera or species.

Actinomycetes propagate by utilizing harmful microorganisms as nutrient sources, thus serving to eliminate the detrimental effects of the harmful microorganisms. Photosynthetic bacteria produce cytokinin and vitamin C precursors promoting cell division, fix atmospheric nitrogen, and further symbiose with azotobacters, which are nitrogen-fixing bacteria found in an environment, thereby promoting a healthy environment. When the environment is soil, the actinomycetes contribute to the fixation of more nitrogen (for example, 3 to 4 times as much nitrogen as usual). Photosynthetic bacteria may also play a role in converting volatile hydrocarbons, hydrogen sulfide, and similar compounds, which are injurious to the roots of crops, to substances useful for the crops, such as carbohydrates, amino acids, and the like. Furthermore, photosynthetic bacteria and certain actinomycetes (such as actinomyces) not only secrete various amino acids useful for the growth of plants but also accumulate such useful amino acids within the cells. Thus, their dead cells serve as a highly effective fertilizer for plants in soil or may serve as food for other syntrophic species of the present disclosure.

When an actinomycete or a photosynthetic bacterium is used alone, it may often fail to defeat harmful microorganisms present in an environment, thereby no longer promoting a healthy environment.

In the present disclosure, a symbiotic relationship between an actinomycete and a photosynthetic bacterium described occurs within the beneficial biofilm of the present disclosure or among the symbiotic progeny of the aforementioned bacteria that have deployed to a recipient environment. These bacteria interact with each other to form a stable system, and their respective functions may be performed efficiently. Moreover, since the aforesaid hydrophilic substrate containing the beneficial biofilm of the present disclosure includes porous bodies, the aforesaid bacteria may be adsorbed in porous regions to form a stable mixed system thereof, where the two form a unique symbiosis within the community of the present disclosure. As a result, not only before the use of the beneficial biofilm but also after its application to a soil, the porous regions may retain the mixed system of the bacteria and provide a base for their action as well as a hub for the incubation and release of progeny into the recipient medium. For example, even when harmful microorganisms are dominant in an environment where the present disclosure is employed, they cannot invade the pores of the porous bodies of the hydrophilic substrate because a stable mixed system of the aforesaid bacteria is formed therein and further the syntrophic system of microbes contained herein is further encased by the BsIA protein coating which is hydrophobic and able to repel harmful microorganisms. Thus, the microbes constituting this mixed system may stay or remain stable in the porous regions and may release progeny. This progeny may migrate out of the confines of the present disclosure and into the surrounding environment and may gradually propagate upon surface pores of hydrophilic substrate materials in the recipient environment, extend their power, and eventually defeat the harmful microorganisms.

Where the beneficial biofilm of the present disclosure contains microorganisms, it utilizes the ability of the microorganism to eliminate the aforesaid detrimental effects of harmful microorganisms from the area to which the beneficial biofilm of the present disclosure is applied and does not utilize certain properties peculiar to any specific strain. In other words, the microorganism used in the beneficial biofilm of the present disclosure as ancillary microbes as described herein are not restricted to a specific strain or a specific pairing. Accordingly, where both a photosynthetic bacterium and an actinomycete are used as ancillary microbes as described here, it is possible to use any desired combination of strains listed above.

The beneficial biofilm of the present disclosure may further contain ancillary microbes, as described herein, other than those described above, and various additives such as specific foodstuff, thereby facilitating the action of the microorganisms added to the present disclosure.

Such ancillary microbes of the present disclosure may include lactic acid bacteria, yeasts, useful mold fungi such as members of the genera Trichoderma and Penicillium, and the like, and such additives include molasses, fish broth, nutrients, and other materials described herein, as well as trace minerals for creating unique enzymes supplied by these microbes.

Lactobacillus acidophilus is the preferred lactic acid bacterium for the present disclosure. Ancillary lactic acid tolerant microbes of the present disclosure may be added to the beneficial biofilm of the present disclosure, but it is preferable to use one which does not induce the formation of butyric acid. Such a lactic acid bacterium may be selected from ones commonly used in lactic acid fermentation, and examples thereof include members of the genus Streptococcus such as S. lactis, S. thermophilus, S. faecalis, S. cremoris, S. diacetilactis, etc.; members of the genus Leuconostoc such as L. mesenteroides, L. dextranicum, L. citrovorum, etc.; members of the genus Pediocuccus such as P. cerevisiae, P. acidilactici, etc.; members of the genus Lactobacillus such as L. bulgaricus, L. acidophilis, L. plantarum, L. delbrukii, L. lactis, L. casei, etc.; and the like. Lactic acid bacteria and Streptococcus members used in the present disclosure are generally regarded as safe (GRAS).

Similarly, Saccharomyces cerevisiae is the preferred yeast for the present disclosure. Ancillary microbes as described herein may include any of various yeasts that may be added to the beneficial biofilm of the present disclosure. Examples of such yeasts include members of the genus Saccharomyces such as S. pastorianus, S. uvarum, S. faecalis, S. fragilis, S. lactis, etc. The Saccharomyces strains used in the present disclosure are generally regarded as safe (GRAS). Additional examples of such yeasts include members of the genus Candida such as C. utilis, C. tropicalis, C. rugosa, C. peculirosa, etc.; and the like. The incubation period for a saccharomyces species useful for the present disclosure varies from about 120 hours to 200 hours depending on the strain selected, the temperature of the system, available oxygen, and the strains of other microbial constituents. S. cerevisiae is comprised of approximately 200 known strains. Those with a faster relative incubation period are selected when formulating a biofilm incubation solution of the present disclosure which is to be used for increasing aeration in soil, increasing nitrogen uptake from the air, and starving or eliminating other microbes. Such varieties may be source from fermented yams from tropical regions.

During the growth of the inoculant, a portion of the Saccharomyces constituent in the present disclosure is consumed by constituent microbes and a portion remains at the end of the incubation period.

When incubation is initiated, B. subtilis completes a single growth cycle in about 48 hours.

Similarly, Lactobacillus acidophilus completes a growth cycle in 25 to 45 hours depending on the strain. L. acidophilus, a cohort of the present disclosure acts to drive down the pH of the biofilm inoculant in the first half of the incubation process which serves to destroy unwanted microbes.

Saccharomyces cerevisiae completes a growth cycle within 120 to 200 hours depending on the strain. S. cerevisiae is a constituent of a common biofilm of the present disclosure for conditioning. S. cerevisiae has numerous phenotypes ranging from the more aerobic, hydrogel forming varieties which consume sugars relatively slow, to the more anaerobic, flagellated types which are known to consume sugars more rapidly and may be more desirable for the present disclosure. The flagella allow them to propel more easily and allows them to make a more extensive account of their surroundings. These flagellated forms seek out hydrophilic substrates, which have pores, valleys, indentations, and other surface variations, which provide an anaerobic environment. Owing to their more rapid consumption of foodstuffs than their hydrogel-forming cohorts, biofilms made with these phenotypes are able to dominate a recipient environment more readily and therefore may lead to greater success in inoculating a recipient environment thereby eliminating the undesirable variant which may already be present as a constituent in a hydrogel, and its potential hazardous hydrogel which may foster algae, slime mold and other unwanted aerobes known to clog drains and drip lines and deplete oxygen in soil, water, the human gut and other environments named herein and yet to be discovered.

Phototrophic bacteria as well as actinomycetes form a symbiotic relationship and may be in a lag phase in the incubation solution until organic acids are created by the presence of other constituents in the system. Organic acids, except butyric acid, may be added to the inoculant and substrate system at any time during the inoculation process in order to enhance the growth of phototrophic bacteria and actinomycetes.

Constituents of the present disclosure may participate in a lag phase before entering into the growth phase depending on temperature, available food sources, and rest after their previous growth period.

Prior to the action of the photosynthetic bacterium and the actinomycete, the lactic acid bacterium functions to secrete a strong acid, thereby destroying harmful microorganisms or weakening their activity. It also functions to convert lignin and tannin, which may hardly be utilized by plants, to useful substances and, in the process, may make iron available for uptake by constituent microbes of the present disclosure.

Generally, photosynthetic bacteria and lactic acid bacteria are anaerobic, actinomycetes are aerobic, and lactic acid bacteria secrete lactic acid which is a strong acid. Accordingly, when a lactic acid bacterium is added to the aforesaid photosynthetic bacterium and an actinomycete, the three may be unable to coexist and propagate together in a liquid medium. However, in the beneficial biofilm of the present disclosure, including the aforesaid combination of bacteria as well as a gas source or air gap, and the useful strain of B. subtilis of the present disclosure and the addition of a hydrophilic substrate, such microorganisms may coexist stably in the beneficial biofilm described herein. As a result, the functions of the respective microorganisms may be fully utilized to produce more excellent effects.

Useful for the present disclosure, Rhodopseudomonas palustris, a photosynthetic bacteria, also known as purple non-sulfur bacteria (PNSB) is known in the art to grow with or without light, fix carbon from organic sources or from carbon dioxide gas, fix nitrogen in the soil that is monatomic from diatomic nitrogen found in the air, use light for energy or use organic or inorganic compound sources for energy. It has been found to enhance the leaf phyllosphere and contribute to the formation and maintenance of plant-growth promoting rhizobacteria. The art explains how PNSB can degrade short chain fatty acids from industrial waste and sewage. PNSB has been found to work in concert with Bacillus subtilis, (another constituent of the present disclosure) to enhance rice yields. PNSB has been found to increase the presence in soil of plant-enhancing microbes such as microbacter. PNSB has been reported to decrease the concentration of salt (NaCl) in aquatic environments such as estuaries.

The yeast, together with the molasses, provides a nutrient source for the aforesaid added microorganisms and also serves to stabilize the mixed system of the aforesaid microorganisms. The molasses is added to the water and dissolved minerals and nutrients and the hydrophilic substrate is added, after this step, the added microorganisms may propagate uniformly and rapidly. Molasses may introduce wild types of saccharomyces which may be useful in some applications of the present disclosure or may be eliminated by first heating the inoculant material containing water, minerals nutrients, and molasses to a temperature and for a duration of time capable of destroying the wild types supplied by molasses.

### The Biofilm Coating

B. Subtilis synthesizes and secretes an exopolysaccharide and an amyloid fiber-forming protein TasA and assembles surface regions with the aid of a small secreted protein BsIA. Bioanalysis demonstrates that BsIA may self-assemble at interfaces, forming elastic regions within the BsIA proteinaceous surface film coating. Natively synthesized and secreted BsIA forms surface layers around constituent microbes of the present disclosure creating an anaerobic beneficial biofilm. The biofilm allows for the progeny of encased microbes to exit the biofilm while denying access to unwanted microbes. The BsIA protein contains localized regions made of non-polar amino acids which are hydrophobic and elastic.

### Method of Action

While not being held to a theory, resident microbes of the present disclosure have undergone considerable changes in phenotype due to the unique biofilm incubation techniques employed and may be at a considerable advantage over their probiotic counterparts which have been grown absent a hydrophilic surface and therefore unable to pair with potentially syntrophic species within a beneficial biofilm. Therefore, the present disclosure may make faster, more complete use of foodstuffs found in a recipient environment than their free-floating probiotic counterparts.

Constituent microbes of the present disclosure are housed within a protective hydrophilic surface and are further encapsulated within a BsIA protein layer. This is not made available to the same species which may be in a free-living probiotic formulation. It is asserted that the environment of the present disclosure allows for the occupants to permanently inhabit the substrate, creating a permanent source of progeny for the recipient environment, and may be modified to release progeny which has been specifically conditioned to gain advantage over competing aerobic systems.

BsIA protein is a product of the present disclosure that is edible and water soluble. As a result, the present disclosure may be used to produce BsIA protein for extraction and use in food products such as ice cream. PNSB, a beneficial constituent of the present disclosure may be increased by submitting the present biofilm system to air, heat and or oxygen towards the end of the incubating process.

### Ingredients of the Inoculant

Unsulfured molasses, black strap molasses, commercial molasses, turbinado, store bought sugar and other commercially available forms of sugars such as candy may be used to create the inoculant in the present disclosure as well as rum and other forms of alcohol. Microfines and volcanic dust as well as rock dust and commercially available forms of trace minerals are also useful in making the inoculant. Bloodmeal and quinoa and other sources of nitrogen such as fish meal may be used. Milk, plain yogurt, sour cream, cheese and related forms of dairy may be used as well as liquid fish sauce, fermented hot sauces, and the wash water from rice. Iron may be supplied by red pepper or paprika as well as minerals and nutrients described herein.

The microbial inoculant composition includes salt (sodium chloride), organic salts, sea salt, Himalayan salt, molybdenum, sea kelp, or mixtures thereof. Suitable amounts of salt, molybdenum, and sea kelp include about 0.001% to about 5%. Any sea kelp known in the art may be used including, without limitation, kelp of the Laminaria, Nereocystis, and Macrocystis genera. Exemplary sea kelps include Laminaria digitata, L. hyperborean, L. ochroleuca, L. saccharina, L. agardhii, L. angustata, L. bongardina, L. cuneifolia, L. dentigera, L. ephemera, L. farlowii, L. groenlandica, L. japonica, L. longicruris, L. nigripes, L. ontermedia, L. pallida, L. platymeris, L. setchellii, L. sinclairii, L. solidungula, L. stenophylla, Alaria marginata, Costaria costata, Durvillea Antarctica, D. willana, D. potatorum, Ecklonia brevipes, E. maxima, E. radiate, Eisena arborea, Egregia menziesii, Hedophyllum sessile, Macrocystis angustifolia, Pleurophycus gardneri, Pterygophora californica, Saccharina japonica, Nereocystis luetkeana, Macrocystis pyrifera, and others known in the art or yet to be discovered.

Examples of useful phototrophic, lactic acid, probiotic, and sulfide-utilizing microorganisms and useful varieties of B. subtilis as well as useful ancillary microbes described herein are found, for example, in Bergey's Manual of Determinative Bacteriology and Bergey's Manual of Systematic Bacteriology. For example, sulfide-utilizing microorganisms useful as ancillary microbes as described herein include species of Purple Non-sulfur Bacteria, Chromatianeae, Green Sulfur Bacteria, Colorless Sulfur Bacteria, and Filamentous Green Bacteria. Probiotic microorganisms may include Lactobacillus genus, Enterococcus genus, Bifidiobacterium genus, Bacillus genus, Pseudomonas genus, Sporolactobacillus genus, Micromonospora genus, Micrococcus genus, Rhodococcus genus, and E. coli. Phototrophic microorganisms may include Rhodopseudomonas, Rodobactor, and combinations thereof. For example, phototrophic microorganisms may include Rhodopseudomonas palustris, R. sphaeroides, Rhodospirillum centenum, R. photometricum, R. rubrum, Rhodopila globiformis, Rhodobacter sphaeroides, and combinations thereof. Lactic acid microorganisms may include Lactobacillus, Lactococcus, and combinations thereof. For examples, lactic acid microorganisms may include Lactobacillus casei, L. plantarum, L. acidophilus, L. fermentum, L. brevis, L. lactis, L. reuteri, L. bulgaricus, L. cellobiosus, L. curvatus, L. delbrukil, L. helbeticus, L. euterii, L. salivarius, L. rhamnosus, L. gaserli, L. jensenii, L. sporogenes, Lactococcus lactis, Streptococcus(Enterococcus ) faecium, S. faecalis, S. cremoris, S. diacetylactis, S. intermedius, S. lactis, S. thermophilus, Pediococuss acidilactici, P. cerevisiae (damnosus), P. pentosaceus, P. acidilacticii, Leuconostoc mesenteroides, and combinations thereof. Bacilli microorganisms may include Bacillus genus and combinations thereof. For example, Bacilli microorganisms may include Bacillus licheniformis, B. subtilus, B. toyoi, B. amyloliquefaciens, B. megateriu, B. pumilus, B. coagulans, B. lentus, B. thermophilus, B. laterosporus, B. cereus, B. circulans, and combinations thereof.

In another embodiment, the composition includes water. Suitable amounts of water include about 1% to about 99% of weight/volume (w/v). Preferably, the amount of water is about 30% to about 99% w/v. More preferably, the amount of water is about 40% to about 95% w/v. Most preferably, the amount of water is about 60% w/v. The composition may be provided in a diluted or concentrated form. In one embodiment, the composition may be a concentrate. In another embodiment, the composition is diluted. In another embodiment, the composition is a slurry made of the inoculant and a portion of the inoculated substrate. In another embodiment, the composition may be a powder and may be pressed with other ingredients into a pill or a pellet.

The compositions of the disclosure may be used for insect control, health supplementation, chemical replacement, soil enrichment, plant enrichment, biodegradation enhancement, food fermentation, carbon sequestration, nitrogen sequestration, aerobic biofilm elimination, food enhancement, and in cleaning solutions. For example, when Rhodopseudomonas palustris (a photosynthetic bacteria named above) is exposed to air, it may fix nitrogen in the soil. The amount of this microbe in the biofilm system may be increased by increasing heat and exposing the system to air. By putting material treated thusly into soil, the soil may then be able to fix nitrogen out of the air and into the soil in a form useful to plants and at levels high enough to replace the need for nitrogen fertilizer. A similar manipulation of the disclosed system may allow for the sequestering of phosphorous and potassium. Such systems, living inside of inoculated charcoal, may become permanent in the soil as long as soil temperatures remain below 110° F. and barring direct exposure to concentrated chemicals which may be harmful.

Methods of the present disclosure include administering a beneficial biofilm that delivers a BsIA containing biofilm of the disclosure to a subject. In one aspect, the composition is orally administered to a subject. In another aspect, the composition is added to food or liquid to be ingested by the subject. The composition may be sprayed on food, in water, or directly in the oral cavity of a subject. The composition may be formulated into a pellet with or without other ingredients contained herein for administering to soil. The formulation may be blended with excipients contained herein to form a pill for oral ingestion, a tincture for treating skin, and a spray to treat surgical devices, to name a few.

Methods of the present disclosure include using a beneficial biofilm to inoculate a substrate thereby producing a biofilm of the present disclosure for use in agriculture practices. In one aspect, the composition is used to enrich soil. The composition may be applied to the soil in liquid, in a slurry comprised of the fully inoculated substrate and the liquid inoculant, or dry in the form of filtered inoculated substrate. Further, fertilizer or other additives may be applied with the composition to the soil. The composition may be applied to the soil surface or mixed into the soil using methods known in the art, such as plowing. The composition may include a subspecies of Saccharomyces cerevisiae cultured from fermented yams from the tropical region of Africa which breaks down sugars at a faster rate than many other subspecies of the same, releasing carbon dioxide gas into the soil, thereby increasing the aeration of the soil, reducing waterborne soil disease and supplying CO₂ for plant uptake. Further, this composition of the present disclosure may be incorporated at a depth of 18 inches into the soil, increasing the ability for air, water, and roots to penetrate and reducing the demand for annual tilling which consumes fuel, thereby reducing the burning of fossil fuels.

In a further embodiment, the inoculation process may include exposing a portion of the inoculant to a light source during its initial creation and allowing increased air intake, light, and/or heat during the inoculation process thereby increasing the ratio of purple non-sulfur bacteria (PNSB) in the present composition. PNSB in combination with actinomyces of the present disclosure has the effect of increasing the available nitrogen in the soil. This occurs by two mechanisms known in the art. Nitrogen contained in plant residue may be sequestered at a higher rate with the addition of the present disclosure and diatomic nitrogen found abundantly in air may be absorbed into the soil, especially during the spring when PNSB lacks a source of energy. One of ordinary skill in the art will appreciate that PNSB readily breaks diatomic nitrogen apart to render the energy from the two nitrogen atoms bonded to one another and may leave the two charged nitrogen particles deposited in the soil for plant uptake and use or for use by the constituent microbes of the present disclosure. Manmade nitrogen fertilizer manufacture may include the burning of natural gas or other fossil fuels and may thus contribute to an increase in the release of CO₂ into the air. This composition of the present disclosure reduces the demand for burning natural gas and other fossil fuels.

In another aspect, the composition may be used to enrich plants. The composition may be applied to soil or a water source of the plant in liquid, slurry, or dry form. Further, fertilizer or other additives may be applied with the composition to the soil or water source of the plant. In another aspect, the composition may be used to preserve cut flowers or plants. The composition may be applied to the soil or to water the cut flowers or plants to preserve them. In another aspect, the composition may be used to enrich seeds prior to planting. The seed may be soaked in an aqueous solution containing the composition. The seed may have affixed to its surface the dried inoculated substrate of the present disclosure. It may be adhered by a number of agents familiar in the art, such as seed surface moisture, a sugar solution, a cellulose solution within a yogurt coating, and other agents familiar to one of ordinary skill in the art.

In another aspect, the composition may be used to enrich the roots of plants. The composition may be applied to soil or a water source of the plant in liquid, slurry, or dry form. The BsIA protein supplied by the hydrophobon which is made present by B. subtilis a constituent of the present disclosure, forms around the root of a plant thereby protecting the root from invasion by unwanted microbial species, especially certain species of fungi such as Pythium, Phytophthora, Rhizoctonia, Fusarium, and Armillaria.

Methods of the present disclosure include using a beneficial biofilm as a chemical replacement. In one aspect, the composition is used as an insecticide or pesticide to increase insect or pest mortality. The composition may be applied to an area in liquid, slurry, pellet, foliar or dry form. Such areas include outside and inside living quarters, as well as directly to the skin of subjects.

Methods of the disclosure also include using beneficial biofilms as cleaning solutions. The composition may be applied to any washable surface or to water. In one aspect, the composition is applied to a body of water such as a pool, pond, lake, lagoon, river, or aquarium. In another aspect, the composition is applied to a hard surface such as concrete, wood, synthetic material, plastic, tile, linoleum, vinyl, fiberglass, composite, glass, granite, marble, or metal. In another aspect, the composition is applied to waste-holding containers such as lagoons, septic tanks, drainpipes, lateral lines, holding tanks, cesspools, and drain fields.

The present disclosure may be placed in a porous capsule capable of containing the substrate material within, and the capsule may be placed in drains, sewage pipes, and other effluent water systems in order to inoculate the wastewater with progeny of the present disclosure continually.

Methods of the disclosure also include using beneficial biofilms in food products made using fermentation. The composition may be applied during the food production process in place of or in conjunction with fermentation agents typically used. The composition may be used in the production of any food requiring fermentation. Examples include, without limitation, beer, wine, cheese, milk, yogurt, kimchi, cider, bread, sauerkraut, sausages, vinegar, pickled foods, kombucha, alcohol, olives, oilseed, chocolate, vanilla, hot sauce, pepperoni, salami, and other foods known in the art or yet to be discovered that rely on fermentation for further enhancement.

Methods of the disclosure also include using the present disclosure in treating garbage from food waste as well as landscape waste and yard waste, often found at landfills. The composition may be applied directly to a mixture of the material and covered with a tarp to ensure a reduced oxygen environment. The resultant material becomes a useful vector for spreading the beneficial microbial constituents. The process enjoys a higher yield of finished product than is found in the process of composting.

### Compositions

In accordance with the present disclosure, inoculant compositions that include ancillary microorganisms, cells, and lysates, as well as methods of use have been discovered. In particular, it has been discovered that a series of related beneficial biofilms may be cultured on substrate material and added to other items and recipient environments for enhancing human health. Such compositions are useful in the agriculture, food, and health industries, as well as chemical replacement in other industries, and may be composed with other ingredients.

Soil and soil amending agents created from the present disclosure may be made using a variety of substrates and methods mentioned herein and may deploy upon a recipient soil found upon the earth and may modify the soil recipient environment, creating a novel composition.

Compositions useful as microbial inoculants for use in this disclosure include microorganisms, substrates, and additives. The microorganisms may include species of bacteria and fungi, including yeast and mold species. Suitable microorganisms include those commonly known in the art as phototrophic, lactic acid, probiotic, and sulfide-utilizing microorganisms as well as some varieties of Bacillus subtilis capable of producing a BsIA hydrophobon surface protein and capable of producing pulcherrimin.

The composition may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the composition is incubated for at least 25 to 65 days. More preferably, the composition is incubated for at least 60 days.

The compositions may also include additives. Suitable additives include substances known in the art that may support growth, production of specific metabolites by the microorganism, alter pH, enrich for target metabolites, enhance insecticidal effects, and combinations thereof. Exemplary additives include carbon sources, nitrogen sources, inorganic salt, organic acid, growth media, vitamins, minerals, acetic acid, amino acids, and the like.

Examples of suitable carbon sources include, without limitation, starch, peptone, yeast extract, amino acids, sugars such as glucose, arabinose, mannose, glucosamine, maltose, sugar cane, molasses, rum, and the like; salts of organic acids such as acetic acid, fumaric acid, adipic acid, propionic acid, citric acid, gluconic acid, malic acid, pyruvic acid, malonic acid and the like; alcohols such as ethanol, glycerol, and the like; oil or fat such as soybean oil, rice bran oil, olive oil, corn oil, and sesame oil. The amount of the carbon source added varies according to the kind of carbon source and is typically between 1 to 100 grams per liter of medium. The weight fraction of the carbon source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition. Preferably, molasses is contained in the medium as a major carbon source, at a concentration of about 2 to 20% (w/v). More preferably, the molasses is at a concentration of about 8 to 12% (w/v).

Examples of suitable nitrogen sources include, without limitation, quinoa, blood meal, blood, amino acids, yeast extract, tryptone, beef extract, peptone, potassium nitrate, ammonium nitrate, ammonium chloride, ammonium sulfate, ammonium phosphate, ammonia, or combinations thereof. The amount of nitrogen source varies according to the nitrogen source, typically between 0.1 to 30 grams per liter of medium. The weight fraction of the nitrogen source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

Examples of suitable inorganic salts include, without limitation, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate, and combinations thereof. The weight fraction of the inorganic salt in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In one embodiment, the compositions of the present disclosure may further comprise alcohol. Suitable alcohols include any known in the art including, without limitation, methanol, ethanol, n-propanol, allyl alcohol, n-propanol, isopropanol, sec-propanol, n-butanol, sec-butanol, isobutanol, t-butanol, and tert-Amyl alcohol. The weight fraction of the alcohol in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions may additionally be provided in a formulation capable of spray. The spray may be a liquid or an aerosol.

The compositions of the present disclosure may also be formulated in a nutritional composition (e.g., foodstuff, food additive, dietary supplement, or feed additive). For example, the compositions may be included in food products made using fermentation techniques such as wine, beer, bread, and cheese.

A nutritional composition of the present disclosure may include any of a variety of nutritional agents, which are well known in the art, including vitamins, minerals, essential and non-essential amino acids, carbohydrates, lipids, foodstuffs, dietary supplements, and the like. Thus, the compositions of the present disclosure may include fiber, enzymes, and other nutrients. Fibers may include, but are not limited to, psyllium, rice bran, oat bran, corn bran, wheat bran, fruit fiber, and the like. Dietary or supplementary enzymes such as lactase, amylase, glucanase, catalase, and the like may also be included. Vitamins for use in the compositions of the present disclosure include vitamins B, C, D, E, folic acid, K, niacin, and the like. Typical vitamins are those recommended for daily consumption and in the recommended daily amount (RDA).

The compositions of the present disclosure may be formulated in a pharmaceutical composition, where it is mixed with a pharmaceutically acceptable carrier for any type of administration route, selected according to the intended use.

In some embodiments, the combination of the disclosure may comprise at least one optional excipient. Non-limiting examples of suitable excipients include antioxidants, additives, diluents, binders, fillers, buffering agents, mineral salts, pH modifying agents, disintegrants, dispersing agents, flavoring agents, nutritive agents, oncotic and osmotic agents, stabilizers, preservatives, palatability enhancers, and coloring agents. The amount and types of excipients utilized to form the combination may be selected according to known principles of pharmaceutical science.

In one embodiment, the excipient may include at least one diluent. Non-limiting examples of suitable diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose.

In still another embodiment, the excipient may comprise a buffering agent. Representative examples of suitable buffering agents include, but are not limited to, 3-(N-morpholino)propanesulfonic acid (MOPS), N-2-hydroxyethylpiperazine-N′-2-ethanesulfonic acid (HEPES), tris(hydroxymethyl)methylamino]propanesulfonic acid (TAPS), Bicine, Tricine, 2-[Tris(hydroxymethyl)methyl amino}-1-ethane sulfonic acid (TES), piperazine-N, N′-bis(2-ethanesulfonic acid (PIPES), 2-(N-morpholino)ethanesulfonic acid (MES), Tris buffers, or buffered saline salts (e.g., Tris buffered saline or phosphate buffered saline).

In a further embodiment, the excipient may include a disintegrant. Suitable disintegrants include, but are not limited to, starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pectin, and tragacanth. In yet another embodiment, the excipient may include a dispersion enhancer. Suitable dispersants may include, but are not limited to, starch, alginic acid, polyvinylpyrrolidones, guar gum, kaolin, bentonite, purified wood cellulose, sodium starch glycolate, isomorphous silicate, and microcrystalline cellulose.

In a further embodiment, the excipient may include a lubricant. Non-limiting examples of suitable lubricants include minerals such as talc or silica; and fats such as vegetable stearin, magnesium stearate, or stearic acid.

In still another embodiment, it may be desirable to provide a coloring agent. Suitable color additives include, but are not limited to, food, drug and cosmetic colors (FD&C), drug and cosmetic colors (D&C), or external drug and cosmetic colors (Ext. D&C).

The weight fraction of the excipient(s) in the combination may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the combination.

The compositions of the present disclosure are stable under various conditions as a liquid or dry form. Preferably, the compositions of the present disclosure are stable at room temperature and may not exceed 110° F. without some likely degradation.

### Methods of Use

The compositions disclosed herein are useful in agriculture, human and animal health, food, and as chemical replacements. The present disclosure encompasses methods of benefiting an environment or subject that would benefit from a beneficial biofilm and eliminating biofilms containing Fusarium oxysporum. The methods may be used to replace chemical compositions, such as insecticides, pesticides, or chemicals. The methods may be used to benefit an environment, such as controlling insect populations, enhancing soil for agriculture purposes, and reducing odor associated with waste. Also, the methods may be used to support or enhance health in a subject. The methods may be used to treat a subject harboring a condition that would benefit from a beneficial biofilm-based therapy or that is at risk of developing a condition that would benefit from beneficial biofilm-based therapy.

### Agriculture

The compositions disclosed herein are useful in agriculture methods. Methods of the disclosure include Fusarium oxysporum biofilm treatment, soil enrichment, plant enrichment, and enhancing biodegradation.

Methods of soil enrichment include applying the beneficial biofilm and inoculated substrate composition to the soil to be enriched. The composition may be in liquid or dry form and applied to the soil by methods known in the art. Exemplary methods include spraying, dropping, scattering, applying pellets, incorporating into the soil during disking, and dusting the target soil. Also, the composition may be applied to a water source that feeds the target soil.

In another aspect, the composition may be used for plant enrichment. Methods of plant enrichment include applying the compositions of the disclosure to the soil or water source of the plant as described herein. Also, the beneficial biofilm and inoculated substrate composition may be added to the water of cut flowers or plants. In another aspect, seeds may be soaked in a composition of the disclosure prior to planting. It may be recognized that it may be beneficial to combine any of the methods described herein for soil and plant enrichment.

The addition of a beneficial biofilm inoculated substrate has the effect of enhancing the biodegradation of various wastes. Such wastes include, without limitation, food waste, waste produced by humans or animals, and landfill waste. A beneficial biofilm also has the effect of enhancing composting. In extraterrestrial environments, the beneficial biofilm system may be modified to break down human wastes such as urine, gas, or feces, as well as minerals found on extraterrestrial surfaces, to produce useful substances for soil building or human consumption.

The beneficial biofilm inoculated substrate may be provided either dried or in liquid form to a waste product. The beneficial biofilm inoculated substrate may be provided in a variety of amounts with respect to the weight of the waste product depending on the waste product. In some aspects, the beneficial biofilm inoculated substrate is provided in an amount ranging from about 0.5 to 50 wt % of the total weight of the waste product. In another aspect, the beneficial biofilm inoculated substrate is provided in an amount ranging from about 1 to about 3 wt % of the total weight of the waste product. In another aspect, the amount of beneficial biofilm inoculated substrate provided to the waste is about 2 wt % of the total amount of waste.

The microorganism may be provided in either dry form, liquid form, or through the spray. In one embodiment, the inoculant may be withdrawn during the incubation and/or fermentation process, and may used for treating plants. Methods of treating waste products include without limitation, spraying, dusting, sprinkling, liquid inoculation, misting, fumigating, aerosolizing, and other methods known in the art.

### Health

Methods of the disclosure include administering the beneficial biofilm inoculated substrate to recipient subjects to support and promote health. Methods of the disclosure include administering compositions to recipient subjects to treat conditions including gastrointestinal and extraintestinal conditions and treating open wounds, ulcers, and surgical incisions. When the beneficial biofilm system is put into an open wound, B. subtillis may supply biofilm constituents that may assist in scab formation, causing a thinner scab. It may also manufacture and release agents known in the art to reduce pain and inflammation, and the biofilm system may deny infections agents.

Examples of gastrointestinal conditions include, without limitation, acute diarrhea, traveler's diarrhea, lactose intolerance, diarrhea associated with human immunodeficiency virus (HIV), sucrose isomaltase deficiency, inflammatory bowel disease, pouchitis, carcinogenesis, enteral feeding-associated diarrhea, antibiotic-associated diarrhea, small bowel bacterial overgrowth, irritable bowel syndrome and conditions associated with enteropathogens. Such enteropathogens include, without limitation, Helicobacter pylori, Campylobacter jejuni, Campylobacter coli, Staphylococcus aureus, Staphylococcus epidermidis, Streptococcus pyogenes, Streptococcus pneumoniae, Enterococcus faecalis, Haemophilus influenzae, Escherichia coli, Klebsiella pneumoniae, Enterobacter cloacae, Citrobacter freundii, Serratia marcescens, Pseudomonas aeruginosa and Pseudomonas maltophilia, Salmonella sp., Gasterophilus sp., Habronema sp., Crascia sp., Trichostrongvlus sp., Parascaris sp., Stroncrulus sp., Triodontophorus sp., Oxvuris sp., Stroncivloides sp., Anonlocephala sp., Paranonlocephala sp., Haemonchus sp., Hvostroncmulus sp., Spirocerca sp., Physoloptera sp., viruses such as rotavirus, fungi such as Candida albicans, Aspergillus fumigatus, Fusarium oxysporum, and other species known or found to be associated with gastrointestinal conditions, and combinations of these species. Also contemplated are pathogens known in the art to cause gastrointestinal conditions such as those described in “Merck's Veterinary Manual” by Cynthia M. Kahn or “The Merck Manual of Diagnosis and Therapy” by Mark H. Beers, both incorporated herein by reference.

Also contemplated, is the use of beneficial biofilms to treat extraintestinal conditions. Without being bound to a theory, extraintestinal conditions may be treated with microorganisms, microorganism extracts, or microorganism products that may stimulate multiple defense mechanisms including promotion of a nonimmunologic gut defense barrier. This barrier may inhibit translocation of potential pathogens and thus prevent infections of the bloodstream and other tissues or organs. Another defense mechanism includes enhancing the intestine's immunologic barrier.

Examples of extraintestinal conditions include, without limitation, appendicitis, autoimmune disorders, multiple sclerosis, Alzheimer's disease, rheumatoid arthritis, celiac disease, diabetes mellitus, organ transplantation, periodontal disease, urogenital diseases (vaginal, urethral and perineal), sexually transmitted disease, HIV infection, HIV replication, surgical associated trauma, surgical-induced metastatic disease, sepsis, weight loss, anorexia, fever control, cachexia, wound healing, ulcers, gut barrier function, allergy, asthma, respiratory disorders, rhinovirus-associated diseases, otitis media, sinusitis, pulmonary disease, circulatory disorders, coronary heart disease, anemia, disorders of the blood coagulation system, renal disease, disorders of the central nervous system, hepatic diseases, constipation, ischaemia, nutritional disorders, osteoporosis, endocrine disorder, epidermal disorders, psoriasis, anthrax, and acne, as well as other conditions known in the art or yet to be discovered that may benefit from treatment with microorganisms, microorganism extracts, or microorganism products.

In use, the beneficial biofilm may be implemented in a number of different ways depending in part on the targeted subjects and the goal of the application. A liquid solution containing a beneficial biofilm inoculated substrate may simply be applied directly into the subject's mouth or onto the food or beverage the subject may consume. For example, an exemplary liquid spray formulation containing a beneficial biofilm inoculated substrate may be sprayed, for example, on the subject's food prior to consumption.

It may be understood that the beneficial biofilm inoculated substrate used may be provided in the form of pure concentrate (100% concentration) or a diluted composition with additional excipients in the dosage form (i.e., the amount of active ingredient in the composition is less than or equal to 99.99%, and the remainder consists of inactive excipients). If diluted, the amount of beneficial biofilm dispensed in the various dosage forms may range from about 1 to 30%, more preferably between about 4 to 8%. One of skill in the art will appreciate that the volume of active component added to the composition may need to be adjusted to account for the dilution and to ensure the end composition comprises the appropriate final concentration of beneficial biofilm. One of skill in the art will also appreciate that the various components of the composition may be provided in a variety of dosage forms including, but not limited to liquid solution or suspension, emulsion, aerosol, slow release matrices, and the like.

A typical concentration range of microorganisms administered is 10³ to 10¹³ cells per day. Preferably, at least about 10⁶, at least about 10⁷, or at least about 10⁸ cells per day are administered. However, it may be appreciated that the number of microbial agents to be administered may vary according to a number of parameters, including the subject's size, type of disorder, and severity of symptoms.

### Food

Methods of this disclosure include using beneficial biofilm inoculated substrates in the preparation of foods that involve fermentation. Suitable foods include those known in the art such as beer, wine, cider, dough-based products, breads, and dairy products. Also contemplated are methods using compositions for preservation techniques.

The beneficial biofilm inoculated substrate may be provided to fermentable foods along with fermentation microorganisms. In some embodiments, the beneficial biofilm inoculated substrate may replace the microorganisms typically used in fermentation. In other embodiments, they may add to the effect of the fermentation microorganisms.

The amount of beneficial biofilm inoculated substrate added to the food product may vary depending on the food product. In some embodiments, the beneficial biofilm inoculated substrate may be provided as a dry powder. In other embodiments, the beneficial biofilm may be provided as a liquid. Dry formulations of the inoculated substrate may be from about 1% to about 99% or more by weight of the composition, while liquid formulations may generally comprise from about 1% to about 99% or more of the beneficial biofilm inoculated substrate by weight.

### Chemical Replacement

Methods of the present disclosure include using inoculated substrate compositions in place of chemicals. Because the inoculated substrate compositions of the present disclosure are uniquely suited for use in a wide variety of chemical-replacement applications such as for insecticides, pesticides, and cleaning solutions, a wide variety of chemicals may be replaced by use of the disclosure, and such replacements are incorporated therein.

### Vector Control

The beneficial biofilm inoculated substrates disclosed herein are particularly useful as insecticides for topical or systemic application to an environment. Such environments include, without limitation, field crops, grasses, fruits and vegetables, lawns, trees, ornamental plants, sand, humans, animals, and other environments that may benefit from insecticide application. The compositions may be formulated for preventative or prophylactic application to an area, and may in certain circumstances be applied to pets, livestock, animal bedding, humans, or in and around farm equipment, barns, domiciles, agricultural facilities, industrial facilities, and other areas that would benefit from insecticide application.

The beneficial biofilms may be applied to an area or environment of the target insect by conventional methods. Such methods include, without limitation, combining the inoculated substrate with suitable water for spraying, dusting, sprinkling, soil soaking, soil injection, seed coating, seedling coating, foliar spraying, aerating, misting, atomizing, fumigating, aerosolizing, and other methods known in the art.

The beneficial biofilm inoculated substrate may be used in consecutive or simultaneous application to an environmental site alone or in combination with one or more additional insecticides, pesticides, chemicals, fertilizers, or other compounds. The inoculated substrate compositions may also be used in conjunction with other treatments such as fertilizers, weed killers, cryoprotectants, surfactants, detergents, insecticidal soaps, dormant oils, polymers, time-release or biodegradable carrier formulations that permit long-term dosing of a target area following a single application of the formulation. Likewise, the formulations may be prepared into edible “baits” or fashioned into insect “traps” to permit feeding or ingestion by a target insect.

Insects repelled by the inoculated substrate of this disclosure may include any member of a large group of invertebrate animals characterized, in the adult state (non-adult insect states include larvae and pupae), by division of the body into head, thorax, and abdomen, three pairs of legs, and, often, but not always) two pairs of membranous wings. This definition therefore includes but is not limited to a variety of biting insects (e.g., ants, bees, black flies, chiggers, fleas, green head flies, mosquitoes, stable flies, ticks, wasps), wood-boring insects (e.g., termites), noxious insects (e.g., house flies, cockroaches, lice roaches, woodlice), and household pests (e.g., flour and bean beetles, dust mites, moths, silverfish, weevils). The beneficial biofilm inoculated substrate compositions of the present disclosure may be effective insect repellents against a wide spectrum of common insect pests, such as those mentioned above, as well as biting insects, wood-boring insects, noxious insects, and household pests, most particularly mosquitoes, sand flies, stable flies, and ticks. The disclosure also includes effectiveness against all stages of invertebrate animals including adult, larvae, and pupae stages.

Regardless of the method of application, the amount of the inoculated substrate or mature inoculant is applied at an insecticidally-effective amount, which may vary depending on such factors as, for example, the specific target insects to be controlled, the specific environment, location, plant, crop, or agricultural site to be treated, the environmental conditions, and the method, rate, concentration, stability, and quantity of application of the composition. The formulations may also vary with respect to climatic conditions, environmental considerations, frequency of application, and severity of insect infestation.

The concentration of insecticidal composition which is used for environmental, systemic, topical, or foliar application may vary widely depending upon the nature of the particular formulation, means of application, environmental conditions, and degree of activity. Typically, the insecticidal composition may be present in the applied formulation at a concentration of at least about 1% by weight and may be up to and including about 99% by weight. Dry formulations of the compositions may be from about 1% to about 99% or more by weight of the composition, while liquid formulations may generally comprise from about 1% to about 99% or more of the composition by weight. As such, a variety of formulations may be prepared, including those formulations that comprise from about 5% to about 95% or more by weight of the composition mix, including those formulations that comprise from about 10% to about 90% or more by weight of the composition. Naturally, formulations may comprise from about 15% to about 85% or more by weight of the composition, and formulations comprising from about 20% to about 80% or more by weight of the composition are also considered to fall within the scope of the present disclosure.

Beneficial biofilm inoculated substrate preparations may generally contain from about 10⁴ to about 10⁸ cells/mg, although in certain embodiments it may be desirable to utilize formulations comprising from about 10² to about 10⁴ cells/mg, or when more concentrated formulations are desired, compositions comprising from about 10⁸ to about 10¹⁰ or 10¹¹ cells/mg may also be formulated.

The insecticidal formulation of the disclosure may be administered to a particular area or environment in one or more applications as needed, with a typical field application rate per hectare ranging on the order of from about 50 g/hectare to about 500 g/hectare of composition, or alternatively, from about 500 g/hectare to about 1000 g/hectare may be utilized. In certain instances, it may even be desirable to apply the composition to a target area at an application rate of from about 1000 g/hectare to about 5000 g/hectare or more of composition. In fact, all application rates in the range of from about 50 g of composition per hectare to about 10,000 g/hectare are contemplated to be useful in the management, control, and killing of target insect pests using such insecticidal formulations. As such, rates of about 100 g/hectare, about 200 g/hectare, about 300 g/hectare, about 400 g/hectare, about 500 g/hectare, about 600 g/hectare, about 700 g/hectare, about 800 g/hectare, about 900 g/hectare, about 1 kg/hectare, about 1.1 kg/hectare, about 1.2 kg/hectare, about 1.3 kg/hectare, about 1.4 kg/hectare, about 1.5 kg/hectare, about 1.6 kg/hectare, about 1.7 kg/hectare, about 1.8 kg/hectare, about 1.9 kg/hectare, about 2.0 kg/hectare, about 2.5 kg/hectare, about 3.0 kg/hectare, about 3.5 kg/hectare, about 4.0 kg/hectare, about 4.5 kg/hectare, about 6.0 kg/hectare, about 7.0 kg/hectare, about 8.0 kg/hectare, about 8.5 kg/hectare, about 9.0 kg/hectare, and even up to and including about 10.0 kg/hectare or greater of composition may be utilized in certain agricultural, industrial, and domestic applications of the insecticidal formulations described herein.

### Cleaning Solutions and Waste Disposal

Cleaning solutions that use compositions of the disclosed inoculated substrate are contemplated. In particular, cleaning solutions that replace all or part of the chemical component typically included in cleaning solutions are contemplated. Methods of cleaning include contacting a surface to be cleansed with a composition of the disclosure. The disclosed system, if manipulated properly, may eliminate odor. Thus the methods of the disclosure may be used to control odor, control disease-causing pathogens, and break down sludge, scum, dirt, grease, and grime. In another aspect, a composition of the disclosure may be added to water to clean the water of impurities. The compositions of the disclosure may be used in cleaning solutions used to clean a variety of surfaces. Such surfaces include all washable surfaces and all hard surfaces, including plastic, fiberglass, wood, concrete, synthetic materials, composite materials, vegetation, fruits, vegetables, and others known in the art. Cleaning solutions including compositions of the present disclosure may also be used to clean water (i.e. ponds, lakes, rivers, aquariums, pools, etc.), septic tanks, holding tanks, and lagoons.

### Target Surfaces

In the applications above, the beneficial biofilm system may be applied to a number of target surfaces as a slurry, as a liquid inoculant, as a dried, powdered inoculant, or combinations thereof. For agricultural applications the target surface may be soil surface, water, air, a leaf, a root, other portions of one or more plants, an animal's alimentary canal, a barnyard, etc. For health applications the target surface may be a toenail, a fingernail, skin, an open wound, the brain, a kidney, the spleen, bone marrow, stem cells, an embryo, harvested human fecal material used for fecal transplant, blood or its various components such as platelets, etc. For food applications the target surface may be a tabletop for food production, food service items, food surfaces such as the rinds of fruit and vegetables, etc. For vector control, cleaning solution, and waste disposal applications, the target surface may be the interior of drip lines, aquariums, a computer keyboard, the contents in a surgical suite a handrail, a toilet lid, a door handle, a floor, discarded food, human feces, animal feces, human or animal surgical or biohazardous waste, animal carcasses, discarded plant material, etc. One of ordinary skill in the art will appreciate that this list is not intended to be all-encompassing or limiting. A target surface listed for one application may also be appropriate as a target surface in an application not listed. Each application may involve target surfaces not listed as well.

### Examples

As various changes could be made in the above compositions and methods without departing from the scope of the disclosure, it is intended that all matter contained in the above description and in the examples given below, shall be interpreted as illustrative and not in a limiting sense.

### Example 1: A 3^{rd}-Order Beneficial Biofilm with PNSB-Enhanced Microorganism Consortium Formulation

The PNSB-enhanced microorganism consortium formulations used herein were made as follows. Purified water was added to a 40-liter mixing tank. The ratio of molasses to inoculant to 0.9% saline water was 1:1:20 v/v/v. 1.95 liters of molasses (Brix 80+5%, pH 5.7+0.5) and 50 grams of bloodmeal was added to the water in the mixing tank. Next, 10 grams of mineral powder (0.15% Mg (as MgO), 0.6% Fe, 0.15% P (as P₂O₅), 3.2% K (as K₂O) was added to the mixture. 60 grams of diatomaceous earth substrate was added that had been pretreated in a 0.9% saline solution. The resulting mixture was brought to a pH of 6.5 using vinegar. The mixture was pumped into a fermentation tank. A second order biofilm system of the present disclosure including only the preferred microbes and without any ancillary microbes as described, thereby containing the preferred forms of Bacillus subtilis, Saccharomyces cerevisiae, Rhodopseudomonas palustris, Lactobacillus acidophilus, and a useful form of Actinomycetes (from the genera Actinomyces as described herein), was added into the mixing tank and blended. Next, the substrate and biofilm inoculant mix was pumped into a fermentation tank equipped with a system for gas exchange as described herein and held at 21° C. Eight liters of air at 29° C. and 1 atmosphere was pumped into the liquid through a tube at the rate of 0.5 liters per day for 16 days. After 40 days, the system was gradually heated to 35° C. over 3 hours and held at that temperature for 10 days. Air was allowed to enter passively by opening a 3 cm air valve for 7 days. The liquid in the mixture began to change from clear to a deep red indicating the growth of the Rhodopseudomonas palustris.The liquid slurry was pumped and filtered. The supernatant was used to treat a nearby pond for mosquitos and other insects. The filtrate was incorporated in the soil to increase nitrogen uptake and eliminate Fusarium oxysporum biofilms. The residual inoculated charcoal remaining in the mixing tank was rinsed using water and pumped into the fermentation tank for use as a 3^{rd}-order progeny. The probiotic mix was incubated for a total of 50 days and had a final pH below 3.6.

### Example 2: A 4^{th}-Order Beneficial Biofilm with PNSB-Enhanced Microorganism Consortium Formulation using Lactobacillus plantarum as an Ancillary Microbe

Purified water was added to a 40-liter mixing tank. The ratio of molasses to inoculant to 0.9% saline water was 1:1:20 v/v/v. 1.95 liters of Molasses (Brix 80+5%, pH 5.7+0.5) and 50 grams of bloodmeal was added to the water in the mixing tank. Next, 10 grams of mineral powder (0.15% Mg (as MgO), 0.6% Fe, 0.15% P (as P₂O₅), 3.2% K (as K₂O) was added to the mixture. 60 grams of diatomaceous earth substrate was added that had been pretreated in a 0.9% saline solution. The resulting mixture was brought to a pH of 6.5 using vinegar. The mixture was pumped into an inoculation tank. The 3^{rd}-order inoculant was supplied in the form noted in Example 1 above and added into the mixing tank and blended. Next, the ancillary microbe Lactobacillus plantarum was supplied by filtering 0.02 liters of liquid from a 0.3-liter container of sauerkraut, and the probiotic mix was pumped into the fermentation tank. The residue remaining in the mixing tank was rinsed using water and pumped into the inoculation tank. Eight liters of air at 23° C. and 1 atmosphere were pumped into the liquid thru a tube at the rate of 0.5 liters per day for 16 days. The probiotic mix was incubated for 51 days at a temperature of 32° C. The resulting 4^{th}-order beneficial biofilm slurry was sprayed onto a newly-harvested cornfield to reduce the lignin and eliminate Fusarium oxysporum biofilms.

### Summary of Additional Embodiments

Disclosed herein is a method for the creation of a beneficial biofilm, and to the creation of a portable device comprising a substrate material coated with or otherwise carrying the beneficial biofilm.

In one embodiment, a beneficial biofilm may be grown on a hydrophilic substrate, including dust or granules having a varied, hydrophilic surface with pits, valleys, vacuoles, indentures, and hollow interiors with access to the substrate exterior.

In one embodiment, a beneficial biofilm may be created by incubating the hydrophilic substrate in a probiotic biofilm containing syntrophic microbes.

In one embodiment, a probiotic microbial inoculant media may be combined with a hydrophilic substrate, and this combination may be inoculated with a group of free-living probiotic microbes, causing the constituent microbes to sense a quorum among the totality of constituent syntrophic microbes, whereupon they enjoin in the process of a communal lifestyle.

In one embodiment, quorum-sensing among constituent syntrophic microbes causes individual microbes within the group to undergo phenotype changes where suites of genes are altered leading to the secretion of substances and or inhabitation of secreted substances containing EPSs.

In one embodiment, a community of anaerobic microbes enjoined in a cohabitation lifestyle within EPS may be formed, known herein as an anaerobic biofilm.

In one embodiment, an anaerobic biofilm may be created and contained upon the available surface of a hydrophilic substrate thereby becoming portable.

In one embodiment, a hydrophilic substrate has undergone incubation allowing a surface coating of anaerobic biofilm whose progeny may be deposited onto a recipient environment.

In one embodiment, a hydrophilic substrate has undergone incubation allowing a surface coating of anaerobic biofilm to be transferred to a recipient environment where the recipient environment is aerobic.

In one embodiment, a hydrophilic substrate is coated with an anaerobic biofilm and is transferred to an aerobic environment to combat a resident biofilm known here as an aerobic biofilm.

In one embodiment, a hydrophilic substrate is coated with an anaerobic biofilm and is transferred to an aerobic environment to combat the presence of an aerobic biofilm by means of releasing progeny into the aerobic environment.

In one embodiment, a hydrophilic substrate is coated with an anaerobic biofilm and is transferred to an aerobic environment to combat the presence of an aerobic biofilm by means of providing progeny into the aerobic environment that competes for foodstuffs.

In one embodiment, a hydrophilic substrate is coated with an anaerobic biofilm and is transferred to an aerobic environment to combat the presence of an aerobic biofilm by means of combating the aerobic biofilm by secreting substances toxic to the aerobic biofilm.

In one embodiment, a hydrophilic substrate is coated with an anaerobic biofilm and is transferred to an aerobic environment to combat the presence of an aerobic biofilm by means of outcompeting the aerobic biofilm for a substrate surface or anaerobic niche on which progeny of the present disclosure may inhabit.

In one embodiment, a hydrophilic substrate is coated with an anaerobic biofilm and is transferred to an aerobic environment to combat the presence of an aerobic biofilm by means of releasing progeny which may populate the recipient environment.

In one embodiment, hydrophilic substrate is coated with an anaerobic biofilm and is transferred to an aerobic environment to combat the presence of an aerobic biofilm by means of releasing progeny which may undergo autolysis, thereby depositing the lysate material upon the recipient environment.

In one embodiment, a hydrophilic substrate is coated with an anaerobic biofilm and is transferred to an aerobic environment to combat the presence of an aerobic biofilm by cellular self-preservation methods and strategies known by anyone in the art.

In one embodiment, a hydrophilic substrate is coated with an anaerobic biofilm and is transferred to a recipient environment that is anaerobic.

In one embodiment, a hydrophilic substrate is coated with an anaerobic biofilm and is transferred to a recipient environment that is anaerobic, where microbes of the present disclosure release progeny.

In one embodiment, a hydrophilic substrate is coated with an anaerobic biofilm and is transferred to a recipient environment that is anaerobic, where progeny of the present disclosure release progeny that discover a hydrophilic surface.

In one embodiment, a hydrophilic substrate is coated with an anaerobic biofilm and is transferred to a recipient environment that is anaerobic, where progeny of the present disclosure release progeny that discover a hydrophilic surface and available foodstuffs.

In one embodiment, a hydrophilic substrate is coated with an anaerobic biofilm and is transferred to a recipient environment that is anaerobic, where microbes of the present disclosure release progeny, discover a hydrophilic surface, and sense a quorum.

In one embodiment, a hydrophilic substrate is coated with an anaerobic biofilm and is transferred to a recipient environment that is anaerobic, where microbes of the present disclosure discover a hydrophilic surface, sense a quorum, and release progeny that undergo changes in phenotype resulting in the release of EPS and other secretions.

In one embodiment, a hydrophilic substrate is coated with an anaerobic biofilm and is transferred to a recipient environment that is anaerobic, where microbes of the present disclosure release progeny that secretes and or inhabits the secreted EPS and other secretions, forming a similar biofilm to that found in the present disclosure.

In one embodiment, a hydrophilic substrate is coated with an anaerobic biofilm and is transferred to a recipient environment that is anaerobic, where microbes of the present disclosure release progeny that sense a quorum, form a similar biofilm to the present disclosure and enjoin in the process of a communal lifestyle.

In one embodiment, a hydrophilic substrate is coated with an anaerobic biofilm and is transferred to a recipient environment to cause the formation of an anaerobic biofilm for the purpose of releasing progeny for occupying the new habitat and forming another biofilm similar to the present disclosure and engage in efforts to deposit secretions and create an environment that attracts progeny to physically enjoin the two habitats.

In one embodiment, a hydrophilic substrate is coated with an anaerobic biofilm and is transferred to a recipient environment to cause the formation of a similar anaerobic biofilm for the purpose of denying the formation of aerobic biofilms.

In one embodiment, a hydrophilic substrate is coated with an anaerobic biofilm and is transferred to a recipient environment where the recipient environment is on human epithelial cells.

In one embodiment, the present disclosure is in a pellet composition for distribution onto a recipient environment.

In one embodiment, the present disclosure is formulated into a pill with other materials such as excipients and fillers or materials familiar to anyone in the art.

In one embodiment, the present disclosure is formulated into a slurry with mature microbial inoculant and inoculated substrate of the present disclosure for use as a topical treatment agent or ointment for treating injuries such as microbial infections, cuts, tumors, burns, and abrasions to the skin and nails. The ointment for such an application may be provided as a slurry of the liquid or supernatant and the filtrate or inoculated substrate.

In one embodiment, the present disclosure is formulated into a slurry with mature microbial inoculant and inoculated substrate of the present disclosure for use as an elixir for treating injuries such as microbial infections, cuts, tumors, burns and abrasions along the alimentary canal including lips, gums, teeth, tongue, jaw, vocal chords, esophagus, stomach, intestines, rectum, anus and buttocks. In one embodiment the present disclosure may be included in suppositories for treatment of the anal and rectal area.

In one embodiment the present disclosure may applied to nails for treatment of fungal infections of the finger and toenails.

In one embodiment, a slurry containing both inoculated charcoal and the supernatant of the biofilm system may be administered to treat systemic candidiasis.

In one embodiment, such a slurry may be administered to treat colitis.

In one embodiment, such a slurry may be administered into the vagina to eliminate yeast infections.

In one embodiment, such a slurry may be administered by direct injection into a leg to treat lymphatic filariasis.

In one embodiment, such a slurry may be administered into a fresh wound to rinse a newly inserted medical device such as a hip joint

In one embodiment, such a slurry may be administered into a fresh wound such as a gunshot, on a battlefield where sterile methods are not available.

In one embodiment, such a slurry may be administered by direct injection to the bloodstream to reduce organ transplant rejection.

In one embodiment, such a slurry may be administered by direct injection to the bloodstream to lower blood pH.

In one embodiment, such a slurry may be administered by topical treatment twice daily to psoriatic skin to reduce inflammation and flaking.

In one embodiment, such a slurry may be administered by direct injection to treat a cancerous tumor or eliminate it.

In one embodiment, such a slurry may be administered by direct application or by injection to an abscessed tooth to reduce infection and swelling.

In one embodiment, such a slurry may be administered by direct injection into a muscle to reduce pain and swelling.

In one embodiment, such a slurry may be inhaled into the lungs to reduce inflammation such as asthma or pneumonia.

In one embodiment, such a slurry may act as a carrier for transporting drugs to be inhaled into the lungs for treatment.

In one embodiment, such a slurry may be administered into the eye to encourage healing and reduce swelling and pain, etc.

The supernatant of the present disclosure may be administered into the eye to encourage healing and reduce swelling and pain after eye surgery.

In one embodiment, the present disclosure is employed to remove residues from biochar. In one embodiment, the present disclosure is employed to submit biochar to air pressure changes to increase pore size, thereby improving the biochar.

In one embodiment, the present disclosure employs a variety of Saccharomyces cerevisiae that ferments sugars quickly in an inoculant that is exposed to additional amounts of air and light and produces a version of the present disclosure that has high levels of phototrophic bacteria and actinomycetes and is therefore able to fix nitrogen in soil.

In one embodiment, human and other waste from a surgical procedure or other unwanted biological event may be contained within a vessel and the biofilm of the present disclosure added thereto. These contents may be allowed to incubate in the presently-disclosed biofilm system. In this manner, the waste material may be isolated and sanitized of unwanted microbes before the material is removed from the surgical room or the hospital and placed into a sewer system or otherwise disposed of.

In one embodiment, a beneficial biofilm product that is a 5^{th}-order biofilm with no ancillary constituents may be taken regularly at the rate of 10 grams per day of slurry with a dose of an over-the-counter probiotic containing microbes of the Bifidobacterium genus to create a 6^{th}-order biofilm within a person's own gut for a digestive system more able to produce short-chain fatty acids known to benefit mental health.

In one embodiment, an n^{th}-order biofilm as disclosed herein, where n is equal to or greater than 5, with no ancillary constituents, may be used as a slurry to treat the exterior of stem cells during their growth cycle to prevent the formation of aerobic biofilms and unwanted microbes.

In one embodiment, an n^{th}-order biofilm as disclosed herein, where n is equal to or greater than 5, with no ancillary constituents, may be used as a slurry to treat the entirety of the exterior of stem cells during their growth cycle to prevent the formation of aerobic biofilms and unwanted microbes.

In one embodiment, an n^{th}-order biofilm as disclosed herein, where n is equal to or greater than 5, with no ancillary constituents, may be used as a slurry to treat the entirety of the exterior of bone marrow cells during their growth cycle to prevent the formation of aerobic biofilms and unwanted microbes.

In one embodiment, an n^{th}-order biofilm as disclosed herein, where n is equal to or greater than 5, with no ancillary constituents, may be used as a slurry and dripped on the cell surface so that the substrate begins to entirely cover an embryo (grown outside the body) during the growth cycle to prevent the formation of aerobic biofilms and unwanted microbes.

In one embodiment, an n^{th}-order biofilm as disclosed herein, where n is equal to or greater than 5, with no ancillary constituents, may be used as a slurry to treat the exterior of stem cells during their growth cycle to prevent the formation of aerobic biofilms and unwanted microbes.

In one embodiment, an n^{th}-order biofilm as disclosed herein, where n is equal to or greater than 5, with no ancillary constituents, may be used as a slurry to treat the epithelia to prevent the formation of aerobic biofilms and unwanted microbes such as candidiasis known as diaper rash

In one embodiment, an n^{th}-order biofilm as disclosed herein, where n is equal to or greater than 5, with no ancillary constituents, may be used as a slurry to treat finger nails and toenails, to prevent the formation of aerobic biofilms and unwanted microbes.

In one embodiment, an n^{th}-order biofilm as disclosed herein, where n is equal to or greater than 5, with no ancillary constituents, may be used as a slurry to treat open wounds to prevent the formation of aerobic biofilms and unwanted microbes.

In one embodiment, an n^{th}-order biofilm as disclosed herein, where n is equal to or greater than 5, with no ancillary constituents, may be used as a slurry to treat the alimentary canal to prevent the formation of aerobic biofilms and unwanted microbes such as dental plaque.

In one embodiment, an n^{th}-order biofilm as disclosed herein, where n is equal to or greater than 5, with no ancillary constituents, may be taken as a 3 gram tablet of filtrate taken orally twice daily to treat the alimentary canal to prevent the formation of aerobic biofilms and unwanted microbes such as leaky gut syndrome in the colon.

The above listed embodiments of the disclosed biofilm may be implemented singly or in combination to perform one or more functions concurrently. Other devices, apparatus, systems, methods, features and advantages of the disclosure are or will be made apparent to one with skill in the art upon examination of the following figures and detailed description. It is intended that all such additional systems, methods, features and advantages be included within this description, be within the scope of the disclosure, and be protected by the accompanying claims.

### Biofilm Portability Study

A study was conducted to determine if a beneficial biofilm grown on treated activated charcoal as described herein would enhance the growth of cannabis indica. Further, the study was designed to show that activated charcoal inoculated with the beneficial biofilm described herein may be extracted from the supernatant mixture, rinsed, dried, and transferred to a recipient environment, thus demonstrating that the microbial constituents of the inoculant solution are portable, i.e., they may be transferred while being contained within the activated charcoal substrate.

### Methods

A common chemovar of cannabis indica known as granddaddy purple was selected due to its genetic stability and it short flowering period (8 weeks). Clones were taken to ensure uniformity for all the plants studied.

Clones were grown in 3-inch by 3-inch by 2.5-inch inorganic rock wool horticultural media cubes for 3 weeks within a closed environment and transplanted into two gallon cloth pots. They were potted in commercially available soil and a vegetative-stage specific nutrient blend (high in nitrogen). Nutrients were given to the plants in equal portions for 5 weeks. At this point, vegetative plants were approximately 27 inches in height. Care was taken to reject plants that did not appear uniform.

The uniform-appearing, 27-inch tall, vegetative plants were transplanted to 10 gallon plastic pots in a commercially available flowering soil mix which supplied the needed nutrients for complete flowering of the cannabis plant. The plants were grown under 1000-watt high pressure sodium vapor lighting. Plants were watered daily with an inline drip system supplying two drip lines to each plant.

A single batch of a beneficial biofilm as described herein was used. It was grown for 3 months. The inoculated charcoal was filtered from the supernatant. A slurry containing 7.5 ounces by weight of the supernatant and 0.5 ounces by weight of the newly filtered and undried inoculated charcoal was combined. The resulting slurry was stirred and added to group B below. The study groups were as follows:
Group A was a control and had no further additives.
Group B had an additional 8 ounces of beneficial biofilm inoculant with the inoculated charcoal slurry mentioned above.
Group C had an additional 8 ounces of dry, filtered charcoal which had been inoculated

with the beneficial biofilm for 3 months. The biofilm-inoculated charcoal was rinsed clean of residual inoculant with filtered water and allowed to dry for 5 days at 80° F. (26.5 C.) prior to its addition to the soil.

After 8 weeks of flowering, the plants were harvested, the flower buds were removed and dried at a temperature of 60° F., for 5.5 days at 70% humidity. The vegetative leaves were removed.

### Results

Group A, the control group, yielded an average 7.22 ounces of dried flower per plant (31 plants total).
Group B, yielded 8.33 ounces of dried flower per plant (30 plants total)
Group C yielded 8.41 ounces of dried flower per plant (31 plants total)

### Conclusion

This studied indicated that the use of the beneficial biofilm system in Group B yielded a substantial increase in flower over Group A. Further, the results of Group C indicates that when the biofilm system was incorporated onto and within the charcoal substrate and administered without the supernatant, it performed similarly to the supernatant and substrate slurry. The results illustrate that the biofilm system will transfer onto and within an activated charcoal substrate and is a useful method for transferring the biofilm system in dry powder form. to a recipient environment, which in this case is soil.

### Detailed Description of the Figures

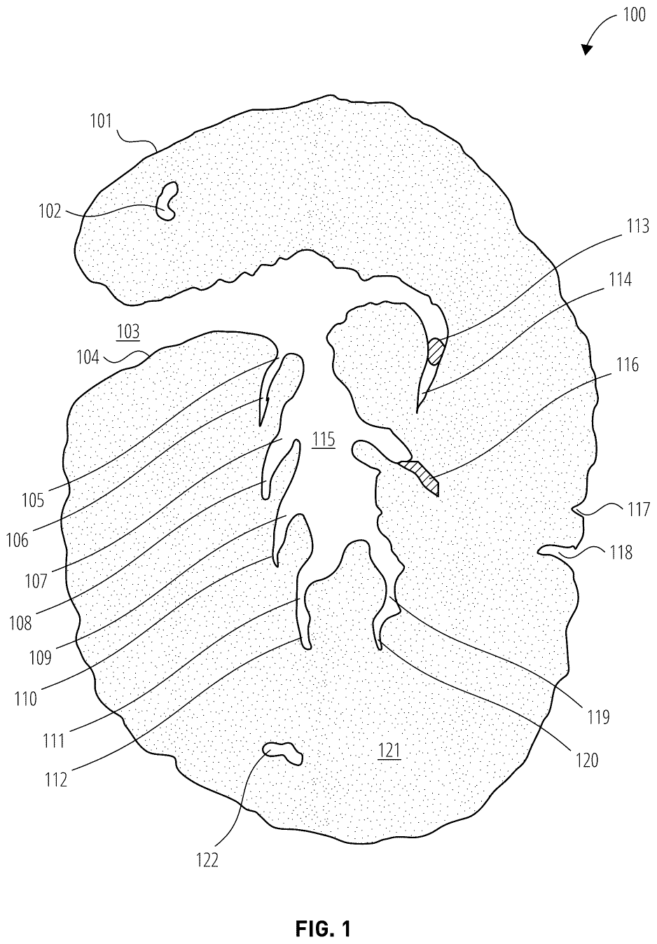

FIG. 1 illustrates a cross-section of one example of an activated charcoal particle 100 in accordance with one embodiment. As illustrated in FIG. 1, an activated charcoal particle 100 may have a varied outer surface 101 containing enclosures, also called closed pores 102 and 122, which do not open to the surface. The charcoal particle may also have a pore structure 103 which has a surface 104.

As used herein, unless specified otherwise, the term “porosity,” “porous,” “porous substrate,” “porous morphology,” and similar terms, such as valley, are to be given the broadest possible meaning, and would include having open pores, closed pores and combinations of open and closed pores, as well as macropores, mesopores, and micropores, and combinations, variations and continua of these morphologies.

The term “solid volume” refers to carbon material and is differentiated from the material void of charcoal, called “free volume,” which is associated with porosity. Activate charcoal may have pores that are blocked by inert debris or substances that are not inert but may or may not contain a portion of inert material such as any combination of the following: chemical agents or residues, biological agents or residues, or inert material.

FIG. 1 illustrates a pore opening along a tunnel, called a macropore 115. The macropore 115 shown opens into smaller pores, such as mesopores 105, 107, 109, and 111. Coursing from macropores to mesopores, a general narrowing occurs into a region termed herein as a micropore. As shown, mesopores 105, 107, 109, and 111 eventually narrow to smaller tunnels or micropores 106, 108, 110, and 112. Micropores 106, 108, 110, and 112 further course down the narrowing tube, and an endpoint occurs in the tunnel regions, ending the free volume region of macropore 115. In some cases, free volume with a continuum of pores may be blocked by an inert particle of debris 113 resulting in a region of free volume 114 which is not connected with the porous network described. A porous region that connects to a continuum of pores may be blocked by inert debris or substances 116 that are not inert but may or may not contain a portion of inert material, such as any combination of the following: chemical agents or residues, biological agents or residues, and inert material.

The activated charcoal particle 100 may have surface pores 117 that do not open into the interior and which may also be termed valleys or other words used in the art to describe impressions or indentures in surface topography. The activated charcoal particle 100 may also have a topography that does not open into the interior which has deeper regions that are the same or similar size as nanopore 118, which may be called surface pores.

A mesopore may become narrow 119 before widening again and as a result may trap air in the accompanying micropore 120. Closed pores 102 that do not open to the surface may occur within the solid volume 121 of charcoal which is contained with the volume of the activated charcoal particle 100.

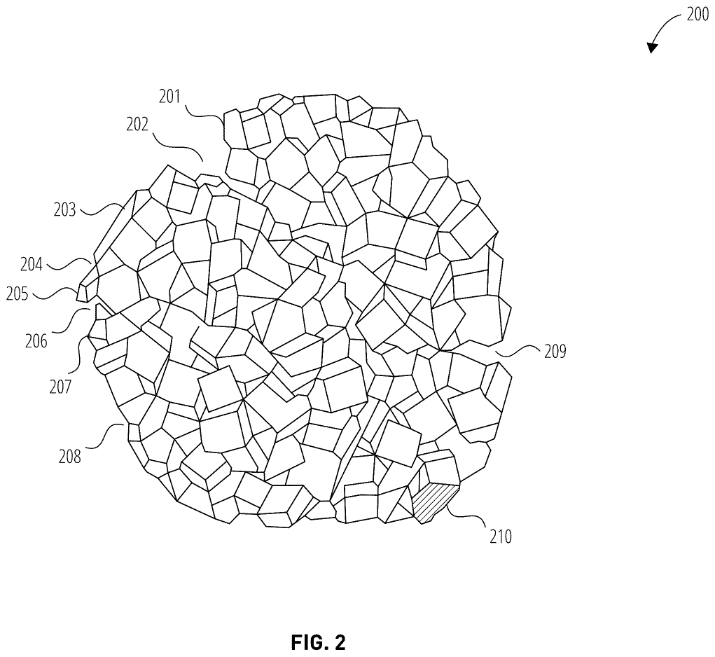

FIG. 2 illustrates a mineral particle hydrophilic substrate 200 in accordance with one embodiment. The mineral particle hydrophilic substrate 200 has a hydrophilic surface 201. The hydrophilic surface 201 of the mineral particle hydrophilic substrate 200 may be hydrophilic and may be made of a relatively pure mineral particle, unground, in crystal form containing a combination of crystal structures having surface structures that project outward, creating valleys of various sizes in the void between.

The exterior-most regions of the mineral particle hydrophilic substrate 200 may contain a solid volume and may have regions projecting outward form the center of the particle. The region between two exterior projecting points constitutes a valley that has a measurable volume of free space. Valleys occurring on the surface topography may connect with one another. The sum of all the valley regions existing upon the topography lying below all exterior projecting points creates a volume of free space.

The mineral particle hydrophilic substrate 200 illustrated has indentures 202. The mineral surface has projections 203, 205, and 207, which point outward from the center of the mineral particle hydrophilic substrate 200. Valleys 204 and 206 occur between these projections. The mineral particle hydrophilic substrate 200 may have surface openings 208 and 209 that are able to trap air. The mineral particle hydrophilic substrate 200 may have on its surface particles of debris 210 that are inert or substances that are not inert but may or may not contain a portion of inert material such as any combination of the following: chemical agents or residues, biological agents or residues, and inert material.

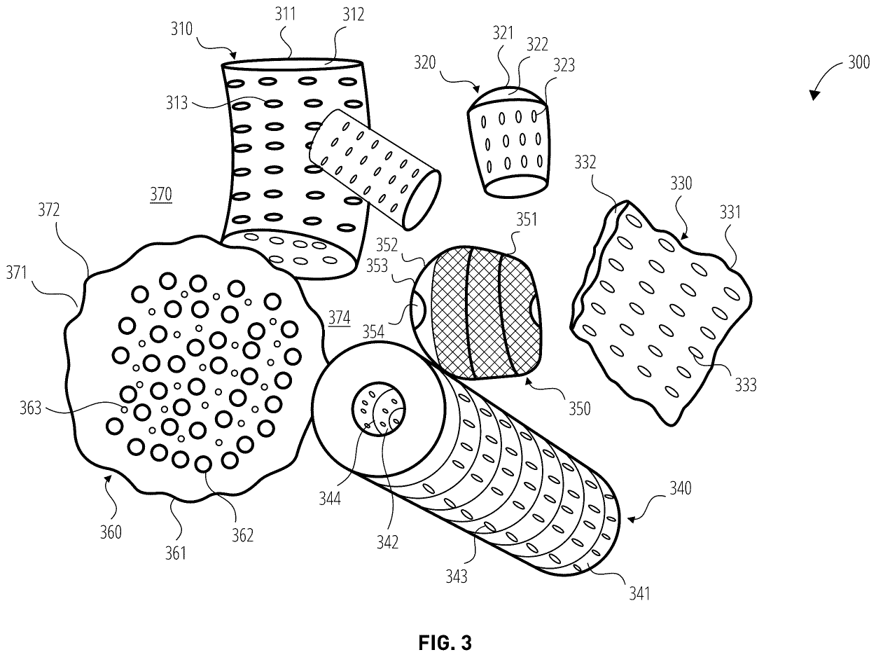

FIG. 3 illustrates diatomaceous earth particles 300, shown at 10,000× magnification through electron microscopy, in accordance with one embodiment. Diatomaceous earth is composed of three-dimensional objects which are known to be hydrophilic. The illustration depicts objects comprising diatomaceous earth such as hypotheca 310, hypotheca 320, intact diatom 350, hypotheca 330, hypotheca 352, hypotheca 340, and saucer-shaped diatom 360, as well as free space 370 among the diatomaceous earth particles 300. Diatomaceous earth (diatomite, celite, kieselgur, or kieselguhr) is a naturally occurring, soft, siliceous sedimentary rock that can be readily crumbled into a fine powder due to its high porosity. It is a collection of diatom shells found in the earth's crust in the form of siliceous sedimentary rock. It has a particle size ranging from more than 3 mm to less than 1 micrometer, but typically 10 to 200 micrometers. It is hydrophilic and is composed of 80-90% silica, with 2-4% alumina and 0.5-2% iron oxide.

Diatomaceous earth is the fossilized remains of diatoms (microscopic single-celled algae) in lake sediment or marine sediments. The fossil remains consist of a pair of symmetrical shells or frustules. The remaining fossilized skeletons of diatoms have a number of different shapes. The most common shape is that of a barrel having numerous pores in rows around the surface. The barrel shape survives most often but is an internal aspect of a paired symmetrical shell system having an internal porous barrel known as the hypotheca which often survives and an outer called an epitheca which readily falls off into small sections. Some varieties develop thick internal barrels or hypotheca which have ridges having pores that open to the outside called pseudoseptum. Diatomaceous earth may also exist in the form of a flat saucer shape having a number of pores. The pores on diatomaceous earth are generally in the range of 250 to 750 nanometers.

In the exemplary diatomaceous earth particles 300 depicted in FIG. 3, diatomaceous earth solid material is made mostly of silica, and hypotheca 310, hypotheca 320, intact diatom 350, hypotheca 330, hypotheca 352, hypotheca 340, and saucer-shaped diatom 360, have surfaces 311, 321, 351, 331, 353, 341, and 361, respectively.

A portion of the diatomaceous earth particles 300 in FIG. 3 are of a barrel shape, known as hypothecae. Hypothecae 310, 320, 330, 352, and 340 have surfaces 311, 321, 331, 353, and 341 and surface openings 312, 322, 332, 354, and 342, respectively. Hypothecae 310, 320, 330, and 340 are shown as having surface pores 313, 323, 333, and 343, respectively. FIG. 3 shows hypotheca 340 as also having interior ridges 344.

Another of the diatomaceous earth particles 300 shown in FIG. 3 is an intact diatom 350 with an exterior layer called an epitheca (shown as epitheca 351) and an interior structure known as a hypotheca (shown as hypotheca 352). The hypotheca 352 has surface 353 and a surface opening 354. FIG. 3 also illustrates a saucer-shaped diatom 360, having a surface 361, large surface pores 362, and small surface pores 363.

FIG. 3 shows free space, including the free space 370 between hypotheca 310 and saucer-shaped diatom 360. Along surface 361 and within the free space 370 between hypotheca 310 and saucer-shaped diatom 360 is an indenture 371. Also shown is a region in the free space 370 where surface 361 includes a protrusion 372 inside of surface opening 312 partially blocking access to the opening of the barrel of hypotheca 310. The free space occurring along surface 361 is shown as free space 373. Additional free space 374 may be found within the barrels of epithecae, as indicated within surface opening 354 of hypotheca 352.

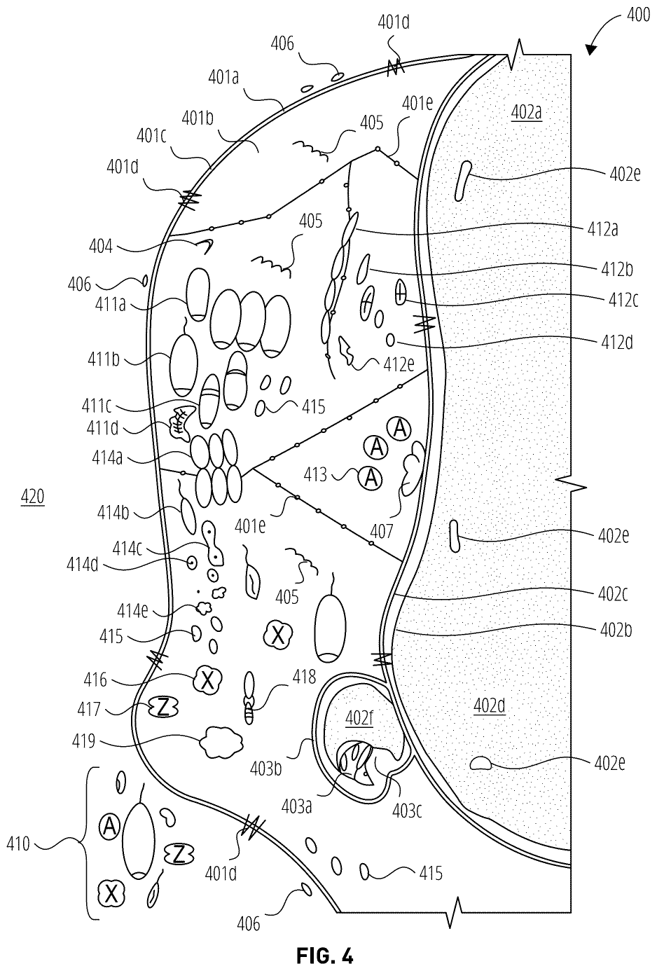

FIG. 4 illustrates a beneficial biofilm 400 in accordance with one embodiment. The beneficial biofilm 400 may have a surface region 401a that forms a enclosure 401b. The surface region 401a may include a BsIA protein coating 401c. The beneficial biofilm 400 may be formed on a hydrophilic substrate 402a. The hydrophilic substrate 402a may also have a surface region 402b that is coated with BsIA protein 402c, formed through the processes disclosed herein.

Solid substrate material 402d of the hydrophilic substrate 402a may include enclosed pores 402e that are not connected to the surface region 402b of the hydrophilic substrate 402a. A small hydrophilic substrate particle 402f may contain its own biofilm matrix 403a. The biofilm matrix 403a may have a surface region 403b, including a BsIA protein coating, around an enclosure 403c. The biofilm matrix 403a may include microbes and excretions of the present disclosure described and depicted herein, all contained within the beneficial biofilm 400 enclosure 401b.

The BsIA may have specific regions made of unique amino acid groupings. Some regions of the beneficial biofilm 400 surface region 401a may be hydrophobic regions 401d made of BsIA protein constructed of non-polar amino acids. TasA protein fibers 401e may be a major structural component within the beneficial biofilm 400. The BsIA protein coating 401c and TasA protein fibers 401e may together form the biofilm enclosure 401b. Within this enclosure 401b there may be small hydrophilic substrate particles 402f, extracellular materials, substances, air bubbles 407, microbes 410, nutrients, and microbial residues as illustrated in FIG. 4 and described below.

The beneficial biofilm 400 may contain exoprotease enzymes 404 and extracellular DNA 405. There may be pulcherrimin 406 on the surface region 401a of the BsIA protein coating 401c. The pulcherrimin 406 may function as a storehouse for iron, is know to be antibacterial, and may limit the size of the beneficial biofilm 400 of the present disclosure.

Bacillus subtilis microbes 410 such as B. subtilis 411a may be a major component of the beneficial biofilm 400, and may provide many of the important components of the beneficial biofilm 400. B. subtilis may also be present in the beneficial biofilm 400 as B. subtilis protozoa 411b, B. subtilis spores 411c, and dead B. subtilis cells 411d.

Lactobacillus acidophilus microbes 410 such as L. acidophilus412a , may be another major component of the beneficial biofilm 400, and may provide organics acids as foodstuffs for other microbes within the present disclosure. L. acidophilus 412a often groups up in end-to-end arrangements as shown, but may also break up to allow individual L. acidophilus 412b to move more readily. L. acidophilus may develop a cleavage 412c when it is ready to divide into newly formed L. acidophilus daughter cells 412d. Dead L. acidophilus cells 412e may also be present in the beneficial biofilm 400.

Actinomyces 413 microbes 410 may be found within the beneficial biofilm 400 enclosure of the present disclosure near a portion of air bubbles 407.

Saccharomyces cerevisiae microbes 410 such as S. cerevisiae 414a may be another major component of the beneficial biofilm 400, and may be a source of foodstuffs for other microbes within the present disclosure. S. cerevisiae protozoa 414b have flagella and may be able to move about more readily than S. cerevisiae's sessile cohort. S. cerevisiae 414a may divide by asexual budding 414c. Newly formed S. cerevisiae daughter cells 414d may become sessile or may form tails. Dead S. cerevisiae cells 414e may also be present in the beneficial biofilm 400.

Phototrophic bacteria 415 microbes 410 may reside near the surface region 401a of the beneficial biofilm 400, as the surface region 401a may be an ideal place for the phototrophic bacteria 415 to gather light, one of several energy sources it may use for its survival.

As described herein, microbes 410 may include ancillary cells that may be added to the inoculant of the present disclosure and may be contained within the beneficial biofilm 400. These may include antagonistic ancillary cells 416 that are antagonistic to the biofilm of the present disclosure. Antagonistic ancillary cells 416 may be added in very small amounts to the inoculant in order to alert the microbes 410 of the beneficial biofilm 400 of the present disclosure that the antagonist may be contained within the recipient environment 420. The beneficial biofilm 400 may also contain supportive ancillary cells 417, which may contribute added enzymes, synthesize foodstuffs, and contribute progeny that is better equipped to combat challenges in the recipient environment 420. Supportive ancillary cells 417 may be added for other reasons that one of ordinary skill in the art will appreciate, or for reasons unforeseen at the present.

Ancillary S. cerevisiae cells 418 may be added that are of a different variety than S. cerevisiae 414a. Two or more variants of S. cerevisiae may be combined in the present disclosure. S. cerevisiae may reproduce both asexually through budding, as illustrated, and sexually. An ancillary lysate additive 419, which may be in the form of dead cells, may be included in the beneficial biofilm 400 in order to add enzymes, foodstuffs, and other cellular components such as pulcherrimin to enhance the present disclosure.

The beneficial biofilm 400 enclosure 401b and the hydrophilic substrate 402a may reside in a recipient environment 420. Microbes 410 of the present disclosure, described herein and illustrated as part of the beneficial biofilm 400, may exit the beneficial biofilm 400 enclosure 401b into or onto the recipient environment 420. The surface regions 401a and 402b may develop openings that allow constituent microbes 410 such as B. subtilis411a , L. acidophilus412a , Actinomyces 413, S. cerevisiae 414a, phototrophic bacteria 415, antagonistic ancillary cells 416, supportive ancillary cells 417, and ancillary S. cerevisiae cells 418 to exit enclosure 401b.

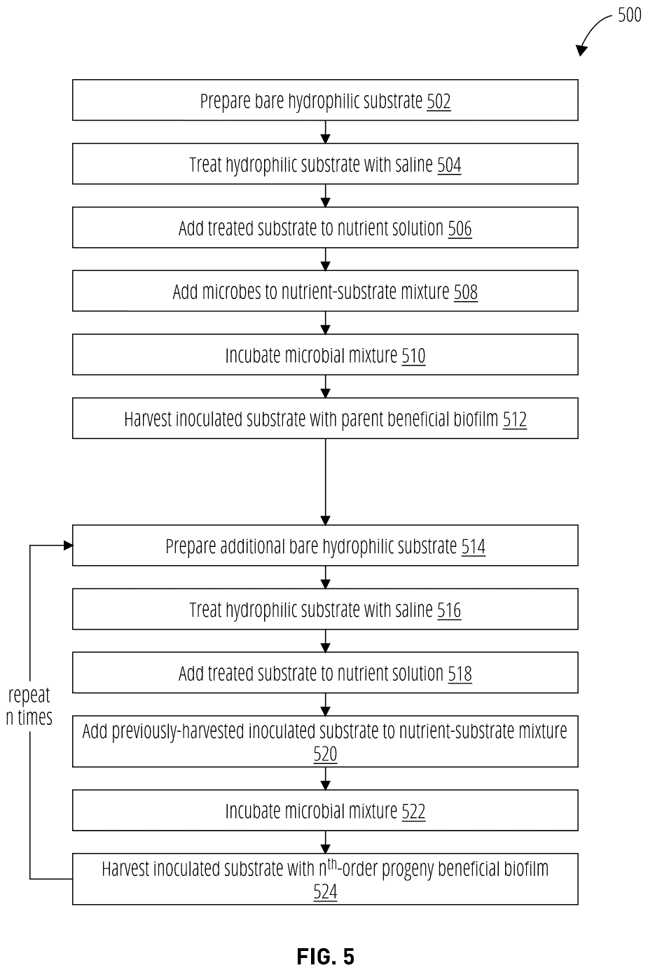

FIG. 5 illustrates a process 500 for creating a pulcherrimin-forming biofilm on a hydrophilic surface in accordance with one embodiment, wherein the hydrophilic substrate may be inoculated with an inoculant, and that inoculated substrate (parent biofilm) may be used as the source of microbes for a secondary inoculation and resulting biofilm and incubation liquid, known as simple progeny. One of skill in the art will recognize that through process 500, an inoculated substrate of any generation (noted herein as n^{th}-order progeny) may be used to create a resultant (n+1)^{th}-order progeny.

Although the example process 500 depicts a particular sequence of operations, the sequence may be altered without departing from the scope of the present disclosure. For example, some of the operations depicted may be performed in parallel or in a different sequence that does not materially affect the function of process 500. In other examples, different components of an example device or system that implements the process 500 may perform functions at substantially the same time or in a specific sequence.

According to some examples, the method includes preparing bare hydrophilic substrate at step 502. In one embodiment this may be an n^{th} bare hydrophilic substrate. According to some examples, the method includes treating the bare hydrophilic substrate with a saline solution at step 504. In one embodiment, the saline solution may be between 0.8 and 1.0 percent sodium chloride by weight. In one embodiment, this may form an n^{th} treated substrate. In one embodiment, n=0. In one embodiment, the hydrophilic substrate may comprise at least one hydrophilic surface including: charcoal, bone, charred bone, diatomaceous earth, coral, limestone, gypsum, particles of trace minerals, hydrophilic metal surfaces including pores, and hydrophilic metal surfaces including valleys.

According to some examples, the method includes adding the treated substrate to the nutrient solution at step 506 to form a nutrient-substrate mixture. In one embodiment, the nutrient solution may be an n^{th} nutrient solution and the nutrient-substrate mixture may be an n^{th} nutrient-substrate mixture. In one embodiment, the nutrient solution may include at least one of: molasses, fish paste, fish emulsion, blood, bloodmeal, fermented shrimp, rice bran, wheat bran, fermented yams, sea salt, granulated kelp, vitamins, soy flour, malic acid, quinoa flour, polysorbate 80, agar, and minerals useful for growing the microbes.

According to some examples, the method includes adding microbes to the nutrient-substrate mixture at step 508. Where the nutrient-substrate mixture is an n^{th} nutrient-substrate mixture, this may form an n^{th} microbial mixture. In one embodiment the microbes may comprise at least a bacillus subtilis that has the ability to perform at least one of the following: synthesize pulcherriminic acid; synthesize and secrete an exopolysaccharide and an amyloid fiber-forming protein TasA; and assemble surface regions with the aid of a bacterial hydrophobon that produces BsIA protein. In one embodiment the microbes may further comprise at least one of phototrophic microbes, lactic acid microbes, probiotic microbes, and sulfide-utilizing microbes. In some embodiments, an air gap may be left when the microbes are added. An exemplary air gap may be 15-20% of the resulting microbial mixture by volume.

According to some examples, the method includes incubating the microbial mixture at step 510. In one embodiment, incubating the microbial mixture occurs in a sealed vessel with an air gap, wherein the air gap includes at least one of oxygen, carbon dioxide, and nitrogen gas. Within the sealed vessel, gas exchange of air, oxygen, carbon dioxide, and nitrogen gas, may be allowed at levels sufficient to supply the growth of all constituent microbes and hindering none.

Incubation may last from 21 to 60 days. During incubation, the mixture may be isolated from light and kept at 90° F., in one embodiment. These conditions may be varied to provide desired results. In one embodiment, where the microbial mixture is an n^{th} microbial mixture, incubation may result in an n^{th}-order progeny inoculated substrate including an n^{th}-order progeny beneficial biofilm.

According to some examples, the method includes harvesting inoculated substrate with parent beneficial biofilm at step 512. Harvesting in one embodiment may include separating the inoculated substrate from the beneficial biofilm. In one embodiment the inoculated substrate is an n^{th}-order progeny inoculated substrate and the parent beneficial biofilm is an n^{th}-order progeny beneficial biofilm.

According to some examples, the method includes preparing additional bare hydrophilic substrate at step 514. According to some examples, the method includes treating the hydrophilic substrate with saline solution at step 516. The bare hydrophilic substrate may be an (n+1)^{th} bare hydrophilic substrate, treatment then resulting in an (n+1)^{th} treated substrate. In one embodiment, n=n+1. Where n previously equaled 0, n may now equal 1, where n previously equaled 1, n may now equal 2, etc.

According to some examples, the method includes adding treated substrate to the nutrient solution at step 518. In one embodiment, the nutrient solution may be an (n+1)^{th} nutrient solution, thus an (n+1)^{th} nutrient-substrate mixture may be formed.

According to some examples, the method includes adding previously-harvested inoculated substrate to the nutrient-substrate mixture at step 520. In one embodiment, the previously-harvested inoculated substrate may be the n^{th}-order progeny inoculated substrate. The addition of the n^{th}-order progeny inoculated substrate to the (n+1)^{th} nutrient-substrate mixture may then form an (n+1)^{th} microbial mixture.

According to some examples, the method includes incubating the microbial mixture at step 522. Air gap and incubation conditions may be as described above or may be varied to provide desired results. Where the microbial mixture is an (n+1)^{th} microbial mixture, the incubation may result in an (n+1)^{th}-order progeny inoculated substrate with an (n+1)^{th}-order progeny beneficial biofilm. According to some examples, the method includes harvesting the inoculated substrate with the beneficial biofilm at step 524. Steps 514 through 524 may be repeated n times to harvest n^{th}-order progeny for increasing values of n. In one embodiment, repeating the process for generating the (n+1)^{th}-order progeny may be performed to generate an (n+1)^{th}-order progeny where n=n+1.

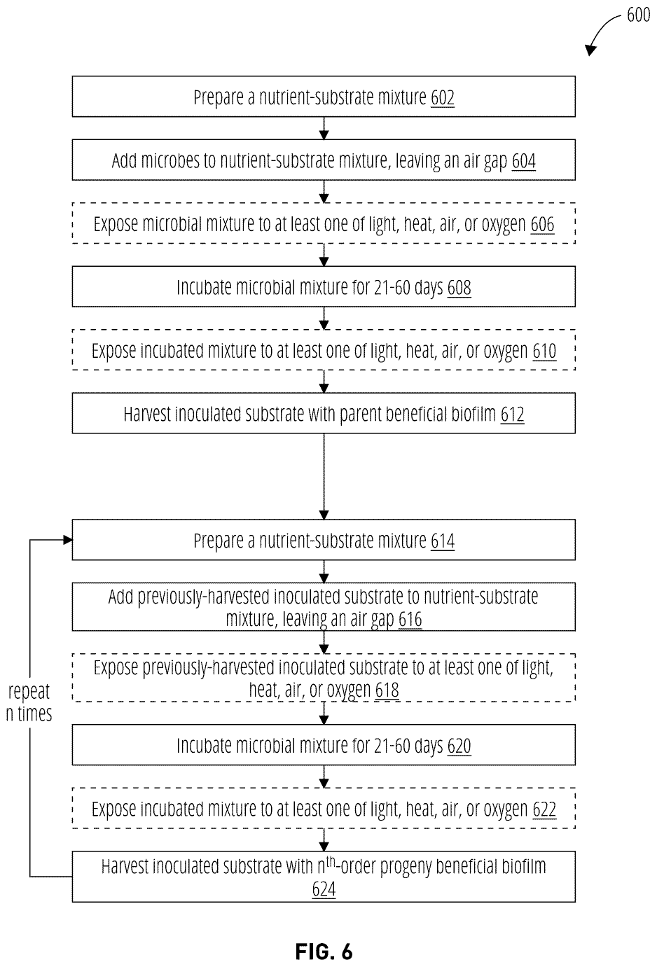

FIG. 6 illustrates process 600 in accordance with one embodiment. Process 600 may build upon process 500, described above, through additional steps of introducing external environmental agents such as exposure to light and/or heat and/or air and/or oxygen. The addition may be done solely with a single causative agent or in any combination. Exposure of the causative agents to the inoculation process is known to increase the amount of Purple Non-Sulfur Bacteria (PNSB) a constituent microbe. Exposure to the mentioned causative agents may also cause modifications in the resulting biofilm-coated substrate and mature incubation liquid material. Beneficial biofilms modified by variations with the causative agents of light, heat, air, or oxygen are known herein as light-modified progeny, heat-modified progeny, air-modified progeny, and oxygen-modified progeny, abbreviated as L mod, H mod, O mod, A mod, respectively. Modifications brought about by the exposure to the mentioned causative agents may be brought about by the use of any combination of causative agents before, during, or after incubation.

FIG. 6 illustrates an example process 600 for creating a pulcherrimin-forming biofilm on a hydrophilic surface. Although the example process 600 depicts a particular sequence of operations, the sequence may be altered without departing from the scope of the present disclosure. For example, some of the operations depicted may be performed in parallel or in a different sequence that does not materially affect the function of process 600. In other examples, different components of an example device or system that implements the process 600 may perform functions at substantially the same time or in a specific sequence.

According to some examples, the method includes preparing a nutrient-substrate mixture at step 602. According to some examples, the method includes adding microbes to the nutrient-substrate mixture, leaving an air gap at step 604. According to some examples, the method includes exposing the microbial mixture to at least one of light, heat, air, or oxygen at step 606. According to some examples, the method includes incubating the microbial mixture for 21-60 days at step 608. According to some examples, the method includes exposing the incubated mixture to at least one of light, heat, air, or oxygen at step 610. According to some examples, the method includes harvesting inoculated substrate with parent beneficial biofilm at step 612.

According to some examples, the method includes preparing a nutrient-substrate mixture at step 614. According to some examples, the method includes adding previously-harvested inoculated substrate to the nutrient-substrate mixture, leaving an air gap at step 616. According to some examples, the method includes exposing the previously-harvested inoculated substrate to at least one of light, heat, air, or oxygen at step 618. According to some examples, the method includes incubating the microbial mixture for 21-60 days at step 620. According to some examples, the method includes exposing the incubated mixture to at least one of light, heat, air, or oxygen at step 622. According to some examples, the method includes harvesting inoculated substrate with n^{th}-order progeny beneficial biofilm at step 624. Steps 614 through 624 may be repeated n times to harvest n^{th}-order progeny.

FIG. 7 illustrates process 700 in accordance with one embodiment. Process 700 may build upon process 500, described above, by including additional material additives such as ancillary microbes that are sympathetic or supportive, antagonistic microbes, and/or lysate.

FIG. 7 illustrates an example process 700 for creating a pulcherrimin-forming biofilm on a hydrophilic surface. Although the example process 700 depicts a particular sequence of operations, the sequence may be altered without departing from the scope of the present disclosure. For example, some of the operations depicted may be performed in parallel or in a different sequence that does not materially affect the function of process 700. In other examples, different components of an example device or system that implements process 700 may perform functions at substantially the same time or in a specific sequence.

According to some examples, the method includes preparing a nutrient-substrate mixture at step 702. According to some examples, the method includes adding microbes to the nutrient-substrate mixture, leaving a 15-20% air gap at step 704. According to some examples, the method includes adding at least one of ancillary microbes, antagonistic microbes, and lysate material to the microbial mixture at step 706. According to some examples, the method includes incubating the microbial mixture for 21-60 days at step 708. According to some examples, the method includes adding at least one of ancillary microbes, antagonistic microbes, and lysate material to the microbial mixture at step 710. According to some examples, the method includes exposing the microbial mixture to at least one of light and air at step 712. According to some examples, the method includes harvesting inoculated substrate with parent beneficial biofilm at step 714.

According to some examples, the method includes preparing additional bare hydrophilic substrate at step 716. According to some examples, the method includes treating the hydrophilic substrate with saline at step 718. According to some examples, the method includes adding treated substrate to the nutrient solution at step 720. According to some examples, the method includes adding previously-harvested inoculated substrate to the nutrient-substrate mixture, leaving a 15-20% air gap at step 722. According to some examples, the method includes incubating the microbial mixture for 21-60 days at step 724. According to some examples, the method includes harvesting inoculated substrate with n^{th}-order progeny beneficial biofilm at step 726. Steps 716 through 726 may be repeated n times to harvest n^{th}-order progeny.

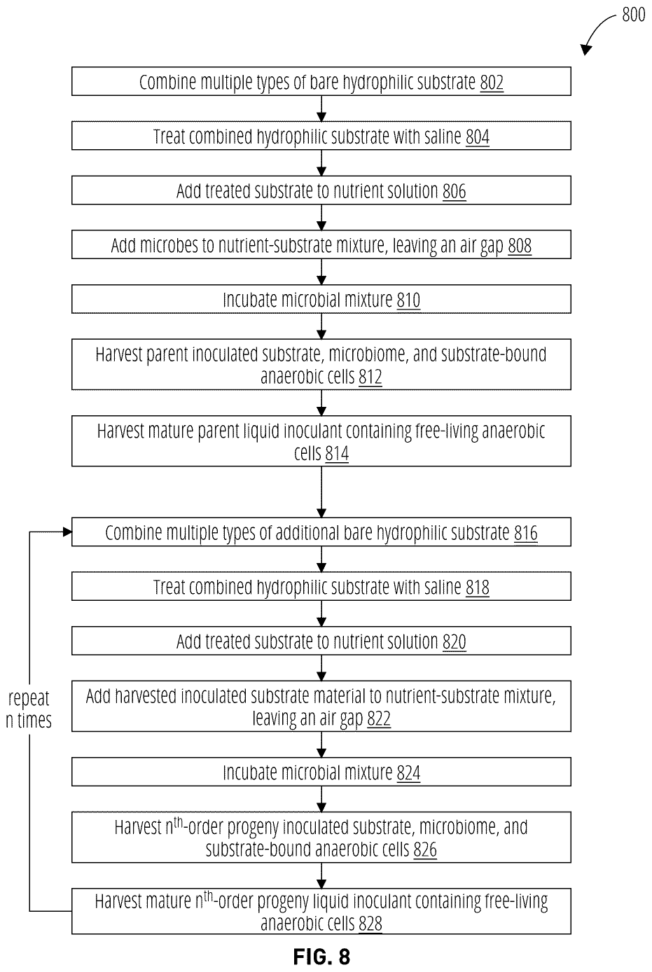

FIG. 8 illustrates process 800 in accordance with one embodiment. Process 800 may build upon process 500, described above, through the additional step of including anaerobic cells other than those used for creating the biofilm as well as the process of modifying the feeding regiment to accommodate those anaerobic cells. The resultant material is known herein as co-cultured progeny.

In one embodiment, the additional anaerobic cells may be bone marrow. The air gap may be less than the normal size and the amount of inoculant of the present disclosure may be of a lesser portion than is normally used. Multiple types and sizes of substrate may be employed in a single inoculating cycle. The substrate used for the anaerobic cells may be bone and it may have relatively large pores, pits, valleys, indentures, and enclosures. The substrate system found in the interior of natural bones is called fossa and may be used to create an environment for growing bone marrow similar to its natural habitat. Additionally, the microbiome of the bone marrow is anaerobic, is able to inhabit hydrophilic substrate material such as bone, and may be harvested with the bone marrow material. Harvested substrate material containing parent bone marrow cells may be reused to grow n^{th}-order progeny bone marrow in successive cycles or may be implanted in a recipient patient. The substrate material used in process 800 may be of a relatively small size and made of a hydrophilic substance other than bone. Other substrate material made of bone may be larger in size in order to accommodate bone marrow cells which are considerably larger than individual microbes of the present disclosure. A screen capable of catching the larger substrate may be employed to separate the two systems during harvest. Through process 800, a technique for delivering a permanent bone marrow culture to the blood may be developed.

FIG. 8 illustrates an example process 800 for creating a pulcherrimin-forming biofilm on a hydrophilic surface. Although the example process 800 depicts a particular sequence of operations, the sequence may be altered without departing from the scope of the present disclosure. For example, some of the operations depicted may be performed in parallel or in a different sequence that does not materially affect the function of process 800. In other examples, different components of an example device or system that implements process 800 may perform functions at substantially the same time or in a specific sequence.

According to some examples, the method includes combining multiple types of bare hydrophilic substrate at step 802. According to some examples, the method includes treating the combined hydrophilic substrate with saline at step 804. According to some examples, the method includes adding treated substrate to the nutrient solution at step 806. According to some examples, the method includes adding microbes to the nutrient-substrate mixture, leaving an air gap at step 808. According to some examples, the method includes incubating the microbial mixture at step 810. According to some examples, the method includes harvesting the parent inoculated substrate, microbiome, and substrate-bound anaerobic cells at step 812. According to some examples, the method includes harvesting mature parent liquid inoculant containing free-living anaerobic cells at step 814.

According to some examples, the method includes combining multiple types of additional bare hydrophilic substrate at step 816. According to some examples, the method includes treating the combined hydrophilic substrate with saline at step 818. According to some examples, the method includes adding the treated substrate to the nutrient solution at step 820. According to some examples, the method includes adding harvested inoculated substrate material to the nutrient-substrate mixture, leaving an air gap at step 822. According to some examples, the method includes incubating the microbial mixture at step 824. According to some examples, the method includes harvesting n^{th}-order progeny inoculated substrate, microbiome, and substrate-bound anaerobic cells at step 826. According to some examples, the method includes harvesting mature n^{th}-order progeny liquid inoculant containing free-living anaerobic cells at step 828. Steps 816 through 828 may be repeated n times to harvest n^{th}-order progeny.

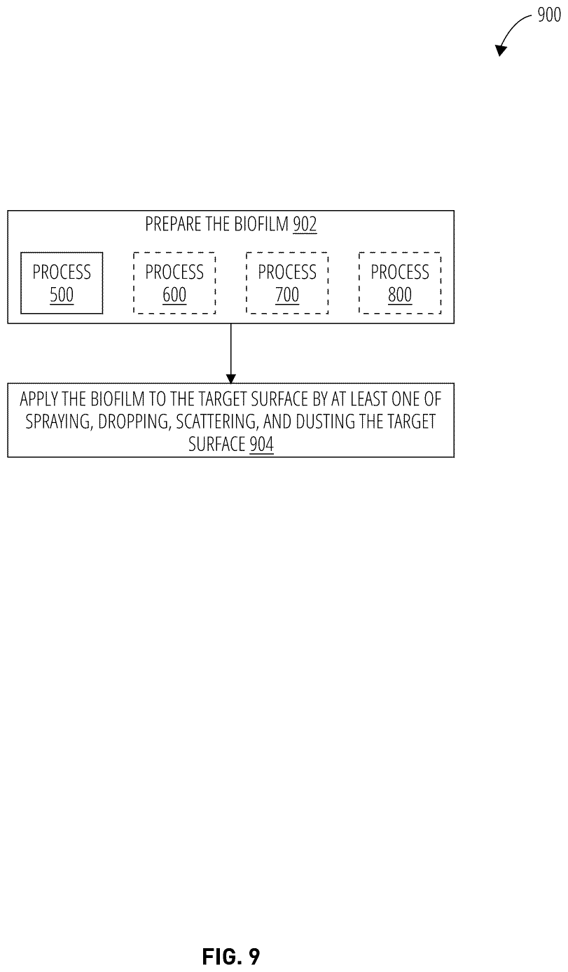

FIG. 9 illustrates an example process 900 for treating a target surface with a biofilm. Although the example process 900 depicts a particular sequence of operations, the sequence may be altered without departing from the scope of the present disclosure.

According to some examples, the method includes preparing the biofilm at block 902. Biofilm preparation may be performed through steps 502-512 of process 500, or through steps 502-524 of process 500 repeated n times. In other embodiments, biofilm preparation may involve some or all of the steps in process 600, process 700, or process 800, singly or in repetition.

According to some examples, the method includes applying the biofilm to the target surface by at least one of spraying, dropping, scattering, and dusting the target surface at block 904. In one embodiment, the target surface may be a soil surface or a plant root. In one embodiment, the target surface may be a toenail or a fingernail. In one embodiment, the target surface may be skin.

When used in the claims, the term “or” is used as an inclusive or and not as an exclusive or. For example, the phrase “at least one of x, y, or z” means any one of x, y, and z, as well as any combination thereof.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

### Definitions

Unless defined otherwise, all technical and scientific terms used herein have the same meaning as is commonly understood by one of ordinary skill in the art. All patents, applications, published applications, and other publications are incorporated by reference in their entirety. In the event that there is a plurality of definitions for a term herein, those in this section prevail unless stated otherwise.

The term “administering” in this disclosure is used in its broadest sense to mean contacting a subject, surface, liquid, or environment with a composition of the disclosed solution.

The term “aerobic biofilm” in this disclosure refers to biofilms commonly found in nature that live in an environment where oxygen is accessible such as in topsoil, the human gut, bloodstream, water troughs, aquariums, agriculture drip lines, and topsoil. The term biofilm is used in the art to describe this common occurrence.

The term “air” in this disclosure refers to ambient air or any combination of gasses used to support specific metabolic processes during the performance of the disclosed processes. Ambient air generally comprises 78 percent nitrogen and 21 percent oxygen, along with small amounts of carbon dioxide, neon, and hydrogen.

The term “air gap” in this disclosure refers to space left in an incubation container that allows for a volume of gas (air) to be present at the start of incubation. Chemical reactions during incubation may change the composition of the gas in the air gap from air, as defined, to contain more carbon dioxide from the incubation process, for example.

The term “anaerobic biofilm” in this disclosure refers to a biofilm that occurs in an environment where oxygen is greatly limited, such as the localized environment on the substrate and within the BsIA hydrophobon protein coating of the present disclosure.

The term “anaerobic cell” in this disclosure refers to a microbe that uses a substance other than oxygen to drive its respiration and metabolic processes.

The term “beneficial biofilm” in this disclosure refers to a biofilm comprising syntrophic microbes in an anaerobic biofilm matrix upon a hydrophilic substrate.

The term “biofilm” used in the art is deficient for describing the present disclosure and needs further explanation, which is provided for by the present descriptive terms: aerobic biofilm, anaerobic biofilm, and beneficial biofilm as used herein.

The term “composition” includes the present disclosure having a beneficial biofilm contained on a hydrophilic surface and may include mature inoculant.

The term “co-culture” in this disclosure refers to a culture of microorganisms that includes at least two microorganisms of the disclosed solution described herein.

The term “excipient” in this disclosure refers to an inert substance added to a pharmaceutical composition to further facilitate administration of a composition. Examples, without limitation, of excipients include calcium carbonate, calcium phosphate, various sugars and types of starch, cellulose derivatives, gelatin, vegetable oils, and polyethylene glycols. Techniques for formulation and administration of pharmaceutical compositions are known in the art and may be found in “Remington's Pharmaceutical Sciences,” Mack Publishing Co., Easton, Pa., latest edition, which is incorporated herein by reference.

The term “facultative end-capping” in this disclosure refers to a process that occurs within the biofilm enclosure where facultative and aerobic microbes of the present disclosure secrete residues and produce progeny that create a protective barrier around anaerobic constituents, protecting them from exposure to air or oxygen.

The term “fermentation product” in this disclosure refers to a mixture including at least one microorganism, expression products of the microorganism(s), substances produced by the microorganisms, and extracts of the microorganisms

The term “finished product” in this disclosure refers to a mixture including an incubated product. The finished product may include additional additives.

The term “free-living anaerobic cell” in this disclosure refers to an anaerobic cell that is not enmeshed in a beneficial biofilm or otherwise attached to substrate material used in the processes disclosed herein.

The term “hydrophilic substrate” in this disclosure refers to a surface that absorbs water when the static water contact angle is less than 90 degrees.

The term hydrophobic substrate in this disclosure refers to a surface that repels water when the static water contact angle is greater than 90 degrees.

The term “insecticidally-effective amount” in this disclosure refers to an amount of the composition that is able to bring about death to at least one insect or noticeably reduce insect growth, feeding, or normal physiological development. This amount may vary depending on such factors as, for example, the specific target insects to be controlled, the specific environment, location, plant, crop, or agricultural site to be treated, the environmental conditions, and the method, rate, concentration, stability, and quantity of application. The formulations may also vary with respect to climatic conditions, environmental considerations, frequency of application, and severity of insect infestation.

The term “microbe” in this disclosure refers to microscopic organisms used to create beneficial biofilms of various compositions, as described herein.

The term “nutrient solution” in this disclosure refers to a liquid solution of compounds that may be metabolized by the microbes introduced via the processes disclosed herein.

The term “pharmaceutical composition” in this disclosure refers to a preparation of one or more compositions of the disclosure with additional components such as physiologically suitable carriers and excipients. The purpose of a pharmaceutical composition is to facilitate administration of a composition to a recipient subject.

The term “physiologically acceptable carrier” in this disclosure refers to a carrier or a diluent that does not cause significant irritation to a subject and does not abrogate the biological activity and properties of the administered composition.

The term “subject” in this disclosure refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific values (e.g., captive or free specimens of endangered species), or mammals which otherwise have value. Suitable subjects also include mice, rats, chickens, rabbits, dogs, cats, ungulates such as cattle, swine, sheep, horses, and goats, lagomorphs such as rabbits and hares, other rodents, and primates such as monkeys, chimps, and apes. Subjects may be of any age including newborn, adolescence, adult, middle age, or elderly.

The term “substrate-bound anaerobic cell” in this disclosure refers to an anaerobic cell attached to the substrate material used in the processes disclosed herein.

Embodiments disclosed herein include:

A. A method of preparing a biofilm, the method comprising: treating an n^{th} bare hydrophilic substrate with a saline solution resulting in an n^{th} treated substrate, wherein n=0; adding the n^{th} treated substrate to an n^{th} nutrient solution to form an n^{th} nutrient-substrate mixture; adding microbes to the n^{th} nutrient-substrate mixture to form an n^{th} microbial mixture; incubating the n^{th} microbial mixture, resulting in an n^{th}-order progeny inoculated substrate with an n^{th}-order progeny beneficial biofilm; harvesting the n^{th}-order progeny inoculated substrate with the n^{th}-order progeny beneficial biofilm.

B. A method for treating a target surface with a biofilm, the method comprising: preparing the biofilm by: treating an n^{th} bare hydrophilic substrate with a saline solution resulting in an n^{th} treated substrate, wherein n=0; adding the n^{th} treated substrate to an n^{th} nutrient solution to form an n^{th} nutrient-substrate mixture; adding microbes to the n^{th} nutrient-substrate mixture to form an n^{th} microbial mixture; incubating the n^{th} microbial mixture, resulting in an n^{th}-order progeny inoculated substrate with an n^{th}-order progeny beneficial biofilm; harvesting the n^{th}-order progeny inoculated substrate with the n^{th}-order progeny beneficial biofilm; and applying the biofilm to the target surface by at least one of spraying, dropping, scattering, and dusting the target surface.

C. A biofilm for treating a target surface, wherein the biofilm is a product of: treating an n^{th} bare hydrophilic substrate with a saline solution resulting in an n^{th} treated substrate, wherein n=0; adding the n^{th} treated substrate to an n^{th} nutrient solution to form an n^{th} nutrient-substrate mixture; adding microbes to the n^{th} nutrient-substrate mixture to form an n^{th} microbial mixture; incubating the n^{th} microbial mixture, resulting in an n^{th}-order progeny inoculated substrate with an n^{th}-order progeny beneficial biofilm; harvesting the n^{th}-order progeny inoculated substrate with the n^{th}-order progeny beneficial biofilm.

Each of the embodiments of A, B, and C may have one or more of the additional elements in any combination: Element 1: wherein the microbes comprise at least a Bacillus subtilis that has the ability to perform at least one of the following: synthesize pulcherriminic acid; synthesize and secrete an exopolysaccharide and an amyloid fiber-forming protein TasA; and assemble surface regions with the aid of a bacterial hydrophobon that produces BsIA protein. Element 2: wherein the microbes further comprise at least one of Rhodopseudomonas palustris, Lactobacillus acidophilous, actinomycetes, and Saccharomyces cerevisiae. Element 3: wherein the hydrophilic substrate comprises at least one hydrophilic surface including: charcoal, bone, charred bone, diatomaceous earth, coral, limestone, gypsum, particles of trace minerals, hydrophilic metal surfaces including pores, hydrophilic metal surfaces including valleys, and hydrophilic extraterrestrial mineral compounds. Element 4: wherein the n^{th} nutrient solution includes at least one of: molasses, fish paste, fish emulsion, blood, bloodmeal, fermented shrimp, rice bran, wheat bran, fermented yams, sea salt, granulated kelp, vitamins, soy flour, malic acid, quinoa flour, polysorbate 80, agar, and minerals useful for growing the microbes. Element 5: wherein the saline solution is between 0.8 and 1.0 percent sodium chloride by weight. Element 6: wherein incubating the n^{th} microbial mixture occurs in a sealed vessel wherein air, oxygen, carbon dioxide, and nitrogen gas are allowed to be exchanged at levels sufficient to supply growth of all of the microbes without hindering the growth of any of the microbes. Element 8: further comprising generating an (n+1)^{th}-order progeny: separating the n^{th}-order progeny inoculated substrate from the n^{th}-order progeny beneficial biofilm; treating an (n+1)^{th} bare hydrophilic substrate with the saline solution, resulting in an (n+1)^{th} treated substrate, wherein n=n+1; adding the (n+1)^{th} treated substrate to an (n+1)^{th} nutrient solution to form an (n+1)^{th} nutrient-substrate mixture; adding the harvested n^{th}-order progeny inoculated substrate to the (n+1)^{th} nutrient-substrate mixture to form an (n+1)^{th} microbial mixture; incubating the (n+1)^{th} microbial mixture, resulting in an (n+1)^{th}-order progeny inoculated substrate with an (n+1)^{th}-order progeny beneficial biofilm; harvesting the (n+1)^{th}-order progeny inoculated substrate with the (n+1)^{th}-order progeny beneficial biofilm. Element 9: further comprising repeating the process for generating the (n+1)^{th}-order progeny to generate an (n+1)^{th}-order progeny where n=n+1. Element 10: wherein the target surface is at least one of a soil surface and a plant root. Element 11: wherein the target surface is at least one of toenail and a fingernail. Element 12: wherein the target surface is skin. Element 13: wherein the hydrophilic substrate comprises at least one hydrophilic surface including: charcoal, bone, charred bone, diatomaceous earth, coral, limestone, gypsum, particles of trace minerals, hydrophilic metal surfaces including pores, and hydrophilic metal surfaces including valleys. Element 14: wherein the biofilm prevents unwanted aerobic biofilms on stents, wherein the biofilm is grown on the stent surface. Element 15: wherein the biofilm prevents unwanted aerobic biofilms on catheters, wherein the biofilm is grown on the catheter surface. Element 16: wherein the biofilm treats colitis, wherein 10 grams of the biofilm is taken each day. Element 17: wherein the biofilm treats herpes simplex one ulcers, wherein a drop of the beneficial biofilm system is placed on the ulcer daily until the ulcer has healed. Element 18: wherein the biofilm prevents and treats infection in a surgical incision, wherein drops of the biofilm are placed on the surgical incision shortly after the surgical incision is closed.

While preferred embodiments of the invention have been shown and described, modifications thereof can be made by one skilled in the art without departing from the teachings of this disclosure. The embodiments described herein are exemplary only, and are not intended to be limiting. Many variations and modifications of the invention disclosed herein are possible and are within the scope of the invention.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method of preparing a biofilm, the method comprising:
treating an n^{th} bare hydrophilic substrate with a saline solution resulting in an n^{th} treated substrate, wherein n=0;
adding the n^{th} treated substrate to an n^{th} nutrient solution to form an n^{th} nutrient-substrate mixture;
adding microbes to the n^{th} nutrient-substrate mixture to form an n^{th} microbial mixture;
incubating the n^{th} microbial mixture, resulting in an n^{th}-order progeny inoculated substrate with an n^{th}-order progeny beneficial biofilm;
harvesting the n^{th}-order progeny inoculated substrate with the n^{th}-order progeny beneficial biofilm.

2. The method of claim 1, wherein the microbes comprise at least a bacillus subtilis that has the ability to perform at least one of the following: synthesize pulcherriminic acid; synthesize and secrete an exopolysaccharide and an amyloid fiber-forming protein TasA; and assemble surface regions with the aid of a bacterial hydrophobon that produces BsIA protein.

3. The method of claim 2, wherein the microbes further comprise at least one of Rhodopseudomonas palustris, Lactobacillus acidophilous, actinomycetes, and Saccharomyces cerevisiae.

4. The method of claim 1, wherein the hydrophilic substrate comprises at least one hydrophilic surface including: charcoal, bone, charred bone, diatomaceous earth, coral, limestone, gypsum, particles of trace minerals, hydrophilic metal surfaces including pores, hydrophilic metal surfaces including valleys, and hydrophilic extraterrestrial mineral compounds.

5. The method of claim 1, wherein the n^{th} nutrient solution includes at least one of: molasses, fish paste, fish emulsion, blood, bloodmeal, fermented shrimp, rice bran, wheat bran, fermented yams, sea salt, granulated kelp, vitamins, soy flour, malic acid, quinoa flour, polysorbate 80, agar, and minerals useful for growing the microbes.

6. The method of claim 1, wherein the saline solution is between 0.8 and 1.0 percent sodium chloride by weight.

7. The method of claim 1, wherein incubating the n^{th} microbial mixture occurs in a sealed vessel wherein air, oxygen, carbon dioxide, and nitrogen gas are allowed to be exchanged at levels sufficient to supply growth of all of the microbes without hindering the growth of any of the microbes.

8. The method of claim 1, further comprising generating an (n+1)^{th}-order progeny:
separating the n^{th}-order progeny inoculated substrate from the n^{th}-order progeny beneficial biofilm;
treating an (n+1)^{th} bare hydrophilic substrate with the saline solution, resulting in an (n+1)^{th} treated substrate, wherein n=n+1;
adding the (n+1)^{th} treated substrate to an (n+1)^{th} nutrient solution to form an (n+1)^{th} nutrient-substrate mixture;
adding the harvested n^{th}-order progeny inoculated substrate to the (n+1)^{th} nutrient-substrate mixture to form an (n+1)^{th} microbial mixture;
incubating the (n+1)^{th} microbial mixture, resulting in an (n+1)^{th}-order progeny inoculated substrate with an (n+1)^{th}-order progeny beneficial biofilm;
harvesting the (n+1)^{th}-order progeny inoculated substrate with the (n+1)^{th}-order progeny beneficial biofilm.

9. The method of claim 8, further comprising repeating the process for generating the (n+1)th-order progeny to generate an (n+1)^{th}-order progeny where n=n+1.

10. A method for treating a target surface with a biofilm, the method comprising:
preparing the biofilm by:
treating an n^{th} bare hydrophilic substrate with a saline solution resulting in an n^{th} treated substrate, wherein n=0;
adding the n^{th} treated substrate to an n^{th} nutrient solution to form an n^{th} nutrient-substrate mixture;
adding microbes to the n^{th} nutrient-substrate mixture to form an n^{th} microbial mixture;
incubating the n^{th} microbial mixture, resulting in an n^{th}-order progeny inoculated substrate with an n^{th}-order progeny beneficial biofilm;
harvesting the n^{th}-order progeny inoculated substrate with the n^{th}-order progeny beneficial biofilm; and
applying the biofilm to the target surface by at least one of spraying, dropping, scattering, and dusting the target surface.

11. The method of claim 10, wherein the microbes comprise at least a bacillus subtilis that has the ability to perform at least one of the following: synthesize pulcherriminic acid; synthesize and secrete an exopolysaccharide and an amyloid fiber-forming protein TasA; and assemble surface regions with the aid of a bacterial hydrophobon that produces BsIA protein.

12. The method of claim 10, wherein the target surface is at least one of a soil surface and a plant root.

13. The method of claim 12, further comprising enhancing the soil surface and underlying soil with monoatomic nitrogen, fixed from diatomic nitrogen in air, as a result of treating the soil surface.

14. The method of claim 12, further comprising increasing an amount of available potassium on the soil surface and underlying soil as a result of treating the soil surface, wherein the soil surface and the underlying soil are infected with a Fusarium oxysporum-including biofilm.

15. A biofilm including a beneficial biofilm inoculant, wherein the beneficial biofilm inoculant is a product of:
treating an n^{th} bare hydrophilic substrate with a saline solution resulting in an n^{th} treated substrate, wherein n=0;
adding the n^{th} treated substrate to an n^{th} nutrient solution to form an n^{th} nutrient-substrate mixture;
adding microbes to the n^{th} nutrient-substrate mixture to form an n^{th} microbial mixture;
incubating the n^{th} microbial mixture, resulting in an n^{th}-order progeny inoculated substrate with an n^{th}-order progeny beneficial biofilm;
harvesting the n^{th}-order progeny inoculated substrate with the n^{th}-order progeny beneficial biofilm.

16. The biofilm of claim 15, wherein the beneficial biofilm inoculant is further a product of at least a Bacillus subtilis that has the ability to perform at least one of the following: synthesize pulcherriminic acid; synthesize and secrete an exopolysaccharide and an amyloid fiber-forming protein TasA; and assemble surface regions with the aid of a bacterial hydrophobon that produces BsIA protein.

17. The biofilm of claim 16, wherein the beneficial biofilm inoculant is further a product of at least one of Rhodopseudomonas palustris, Lactobacillus acidophilous, actinomycetes, and Saccharomyces cerevisiae.

18. The biofilm of claim 15, wherein the hydrophilic substrate comprises at least one hydrophilic surface including: charcoal, bone, charred bone, diatomaceous earth, coral, limestone, gypsum, particles of trace minerals, hydrophilic metal surfaces including pores, hydrophilic metal surfaces including valleys, and hydrophilic extraterrestrial mineral compounds.

19. The biofilm of claim 15, wherein the beneficial biofilm inoculant includes (n+1)^{th}-order progeny and is further a product of:
separating the n^{th}-order progeny inoculated substrate from the n^{th}-order progeny beneficial biofilm;
treating an (n+1)^{th} bare hydrophilic substrate with the saline solution, resulting in an (n+1)^{th} treated substrate, wherein n=n+1;
adding the (n+1)^{th} treated substrate to an (n+1)^{th} nutrient solution to form an (n+1)^{th} nutrient-substrate mixture;
adding the harvested n^{th}-order progeny inoculated substrate to the (n+1)^{th} nutrient-substrate mixture to form an (n+1)^{th} microbial mixture;
incubating the (n+1)^{th} microbial mixture, resulting in an (n+1)^{th}-order progeny inoculated substrate with an (n+1)^{th}-order progeny beneficial biofilm;
harvesting the (n+1)^{th}-order progeny inoculated substrate with the (n+1)^{th}-order progeny beneficial biofilm.

20. The biofilm of claim 19, wherein the beneficial biofilm inoculant is further a product of repeating the process for generating the (n+1)^{th}-order progeny to generate an (n+1)^{th}-order progeny where n=n+1.
